(19) United States
(12) Patent Application Publication
(10) Pub. No.: US 2015/0320706 A1
(43) Pub. Date: Nov. 12, 2015

(54) FORMULATIONS AND METHODS OF TREATING ALZHEIMER'S DISEASE AND OTHER PROTEINOPATHIES BY COMBINATION THERAPY

(71) Applicant: CHIESI FARMACEUTICI S.P.A., Parma (IT)

(72) Inventors: Bruno Pietro IMBIMBO, Parma (IT); Daniel CHAIN, Parma (IT)

(73) Assignee: CHIESI FARMACEUTICI S.P.A., Parma (IT)

(21) Appl. No.: 14/707,033

(22) Filed: May 8, 2015

Related U.S. Application Data
(60) Provisional application No. 61/991,684, filed on May 12, 2014.

(30) Foreign Application Priority Data
May 12, 2014 (EP) 14167880.5

(51) Int. Cl. A61K 31/192; A61K 45/06; A61K 47/42; A61K 39/395; C07K 16/18; A61K 9/00; A61K 31/05

(52) U.S. Cl. CPC A61K 31/192; A61K 9/0053; A61K 45/06; A61K 31/05; A61K 39/3955; C07K 16/18; A61K 47/42; C07K 2317/92; C07K 2317/76; C07K 2317/24; A61K 2039/505

(57) ABSTRACT

Administration of a 1-phenylalkanecarboxylic acid before, after, or a concurrently with a therapeutically effective amount of one or more of the following: (1) β-amyloid peptides level reducers, (2) pathogenic level tau reducers, (3) microtubule stabilizers, (4) agents capable or removing atherosclerotic plaques, (5) agents that lower circulating levels of β-amyloid and tau, (6) modulators of autophagy, (7) neurotransmitter levels regulators, (8) GABA(A) α5 Receptor Antagonists, and (9) additional agents that help maintain and/or restore cognitive function and functional deficits of AD, and/or slow down decline in cognitive functions and functional deficits in AD, is useful for prevention or therapeutical treatment of proteinopathies and/or neurodegenerative diseases.

### CROSS REFERENCES TO RELATED APPLICATIONS

This application claims priority to U.S. Provisional Application No. 61/991,684 filed on May 12, 2014, and European Patent Application No. 14167880.5, filed on May 12, 2014, all both which are incorporated herein by reference in their entireties.

### BACKGROUND OF THE INVENTION

1. Field of the Invention

The present invention relates to formulations and methods for treating Alzheimer's disease and other proteinopathies by combination therapy.

2. Discussion of the Background

Neurodegenerative diseases such as Alzheimer's disease (AD), Parkinson's disease (PD), Huntington's disease (HD), amyotrophic lateral sclerosis (ALS), prion disease, Familial Amyloid Polyneuropathy (FAβ), inclusion body myositis (IBM) and various forms of retinal degeneration such as age related macular degeneration (AMD) are increasingly seen as disorders of protein folding and/or protein aggregation and collectively referred to as proteinopathies. Proteinopathies also include diseases affecting peripheral tissues. They all share some common molecular mechanisms which may lead to microglial impairment, inflammation, protein aggregation, oxidative stress, and/or irreversible tissue damage and ultimately death of nerve cells in an affected subject.

The aggregates in these proteinopathies typically consist of fibers containing misfolded protein with a β-sheet conformation. Examples of proteins that become misfolded resulting in proteinopathies are β-amyloid (AD, cerebral β-amyloid angiopathy, inclusion body mysositis, retinal ganglion degeneration in glaucoma and AMD), microtubule associated protein (multiple tauopathies), α-synuclein (PD), huntingtin (Huntington's disease), prion proteins (multiple prion diseases), TDP-43 (frontotemperal lobar degeneration), superoxide dismutase and FUS (ALS), cystatin C (hereditary cerebral hemorrhage), Notch3 (CADASIL), glial fibrillary acidic protein (Alexander disease), seipin (Seipinopathies), transthyretin (familial amyloidotic neuropathy and senile systemic amyloidosis, monoclonal immunoglobin light chains (AL amyloidosis), monoclonal immunoglobin heavy chain (AH heavy chain amyloidosis), amyloid A protein (AA secondary amyloidosis), islet amyloid polypeptide (Type II diabetes), medin (Aortic medial amyloidosis), apolipoprotein AI (ApoAI amyloidosis), apolipoprotein AII (ApoAII amyloidosis), apolipoprotein AIV (ApoAIV amyloidosis), gelsolin (familial amyloidosis), lysozyme (fibrinogen amyloidosis), beta-2-microglobulin (dialysis amyloidosis), crystallins (cataracts), rhodopsins (retinitis pigmentosa with rhodopsin mutations, calcitonin (medullary thyroid carcinoma), atrial natriuretic factor (cardiac atrial amyloidosis), keratoepithelin, keratins (cutaneous lichen amyloidosis), prolactin (pituitary prolactinoma), lactoferrin (corneal lactoferrin amyloidosis), surfactant protein (pulmonary alveolar proteinosis), semenogelin 1 (seminal vesicle amyloid), CFTR protein (cystic fibrosis) and hemoglobin (sickle cell disease). Amyloid or other misfolded protein aggregates are highly resistant to degradation. For example, β-amyloid deposits, once formed, are stable even in the absence of ongoing amyloid production. In certain cases, amyloid or other misfolded protein aggregates catalyze the structural conversion of the normally folded protein into additional aggregates via a seeded nucleation-dependent process.

In AD, the amyloid β peptide (Aβ) and the microtubule-associated protein tau, are both implicated in pathophysiology with Aβ accumulation in the brain causing pathological changes to tau. A key function of the tau protein is to stabilize microtubules. Microtubules are abundant in neurons of the central nervous system (CNS) and are also expressed at very low levels in CNS astrocytes and oligodendrocytes. Their concentrations are lower outside the CNS. When tau proteins are defective, and no longer stabilize microtubules properly, they can cause and/or contribute to diseases such as, e.g., AD and FTD.

As tau aggregates accumulate, the neuron is further sensitized to Aβ induced toxicity—essentially creating a feedback loop whereby increasing concentrations of pathological tau and Aβ push one another to become even more active. This leads to greater aggregation of tau and amyloid beta and the eventual loss of synaptic function and subsequent neuronal death.

In non-AD dementias, e.g, FTD, mutations in the tau protein can also have profound pathophysiological effects that cause dementia.

Inflammation associated with amyloid accumulation and with over-sensitized or dysfunctional microglia provides a common thread helping to drive pathology in proteinopathies.

In the AD brain, inflammatory response includes, e.g., activated microglia and reactive astrocytes. Activated microglia may mediate neuronal damage by producing toxic cytokines (e.g., TNF-α, IL-1β, etc.), excitatory amino acids and reactive oxygen intermediates. However, microglia can also be neuroprotective, e.g., by clearing β-amyloid through phagocytosis. Based on this dual activity profile, microglial function has been divided into an inflammatory (M1) and a phagocytic (M2) phenotype. During the early phases of AD, initial deposition of Aβ is believed to shift the equilibrium of microglia from the M2 phagocytic to the M1 inflammatory phenotype (Gandy S. et al., Biol. Psychiatry (2013) 73: 393-395, which is incorporated herein by reference in its entirety). The recent discovery that a defective mutation of a microglial phagocytic protein involved in the phagocytic function of microglia, (TREM2) is associated with a threefold increase in the risk of AD (Neumann H and Daly M J. N Engl J Med (2013) 368: 182-184, which is incorporated herein by reference in its entirety) has renewed interest in anti-inflammatory drugs that may fine tune microglial activity by stimulating M2 phagocytic activity and simultaneously inhibiting M1 inflammatory activity (microglial modulators). Another microglial cell-surface protein (CD33) has been genetically linked to AD (Naj et al., Nat Genet. (2011) 43: 436-441, which is incorporated herein by reference in its entirety) and has been recently found in high amounts in the AD brain (Griciuc et al., Neuron (2013) 78:631-643, which is incorporated herein by reference in its entirety), suggesting that disregulation of this protein also plays a role in disease pathogenesis. Other recent studies also link microglia to AD via complement component receptor-1 (CR1 or CD35). Single nucleotide polymorphisms in CR1 were reported to be associated with greater risk of AD (Lambert et al., Nat. Genet. (2009) 41: 1094-1099, which is incorporated herein by reference in its entirety). The rs6656401A risk allele of CR1 has also been related to greater cognitive decline over time in older individuals (Chibnik et al., Ann Neurol (2011) 69: 560-569, which is incorporated herein by reference in its entirety). More recently, it has been shown that loss of CR1 modulates the impact of the apolipoprotein E ε4 (APOE ε4) allele on brain fibrillar amyloid burden, further supporting the concept that microglial dysfunction is important in AD (Thambisetty et al., Biol Psychiatry (2013) 73: 422-428, which is incorporated herein by reference in its entirety).

Therapeutic strategies currently under study for AD and/or other neurodegenerative disorders due to proteinopathy are diverse. For Aβ they include passive administration of antibodies to various conformations of Aβ and vaccines eliciting such antibodies; protease inhibitors and/or modulators targeting the peptide's synthetic enzymes; small molecule amyloid and clearing agents including, e.g., aggregation inhibitors, microtubule stabilizers, PPAR-gamma agonists, antioxidants, anti-inflammatories and compounds targeting additional mechanisms, e.g., neurotransmitter modulation. Strategies are being tested in well over 100 clinical trials, including some involving late stage trials. However, although results from preclinical work have often been promising, results from human clinical trials of many drugs have failed to produce significant clinical benefit and for some have produced significant adverse effects such as meningoencephalitis. Taken together, the results of clinical trials in AD indicate the need for both earlier intervention and new therapeutic strategies.

It is increasingly apparent that monotherapy targeting a single pathological process may not effectively treat complex diseases such as AD and other proteinopathies. For example, where a cascade leading to neurodegeneration is underway, merely removing the initial trigger for the cascade (e.g. β-amyloid accumulation) may not be sufficient to stop the cascade. Similarly, if β-amyloid concentrations are several-fold above those capable of causing neuronal degeneration, a marked reduction in levels alone might be insufficient to slow degeneration. Instead, the ideal scenario might involve administration of β-amyloid lowering treatments in the earliest stages of β-amyloid accumulation, i.e. years before onset of symptoms. This approach would require drugs of exceptionally low toxicity administered with difficulty to achieve high compliance rates years before clinical manifestations begin. In addition, amyloid-based monotherapies are unlikely to improve function or plasticity of previously damaged but surviving neurons. Moreover, amyloid pathology-associated proteins such as apolipoprotein E4 can increase the pathogenicity of the amyloidogenic protein either by increasing the rate of fibrillogenesis or by other mechanisms; thus, treatments for these targets could also be required to achieve maximal effect. Finally, although the bulk of current evidence points to amyloid beta accumulation as a critical primary causative factor in AD, a number of other mechanisms might constitute important causative factors as well. Such non-amyloid beta mechanisms, such as those associated with abnormal tau protein, might play additive or synergistic roles as the disease progresses. Thus, parallel neuroprotective strategies might play a valuable, even a vital, role in delaying AD and other proteinopathies and slowing disease progression. It is therefore likely that the successful treatment of such diseases will require administration of a combination of therapeutic agents.

Although tremendous advances are being made in understanding mechanisms driving neurodegenerative diseases such as AD, PD and other proteinopathies, there is a great unmet need for effective treatments. Agents that can reduce neuroinflammation and/or promote clearance of toxic amyloid proteins such as amyloid beta and tau proteins could be valuable and effective treatments for such diseases.

Several epidemiological studies suggest that long-term use of non-steroidal anti-inflammatory drugs (NSAIDs) may protect subjects carrying one or more ε4 allele of the apolipoprotein E (APOE ε4) against the onset of AD. The biological mechanism of this protection is not completely understood and may involve the anti-inflammatory properties of NSAIDs or their ability to interfere with the Aβ cascade. Unfortunately, long-term, placebo-controlled clinical trials with both non-selective and cyclooxygenase-2 (COX-2) selective NSAIDs in mild-to-moderate AD patients produced negative results. A secondary prevention study with rofecoxib, a COX-2 selective inhibitor, in patients with mild cognitive impairment (MCI) was also negative. A primary prevention study (ADAPT trial) of naproxen (a non-selective COX inhibitor) and celecoxib (a COX-2 selective inhibitor) in cognitively normal elderly subjects with a family history of AD was prematurely interrupted for safety reasons after a mean period of treatment of 2 years. Although neither drug reduced the incidence of dementia after two years of treatment, surprisingly, a 4-year follow-up assessment revealed that subjects previously exposed to naproxen were protected from the onset of AD by 67% compared to placebo. Thus, it could be hypothesized that the use of classic NSAIDs may be beneficial only in the very early stages of the AD process in coincidence with initial Aβ deposition, microglia activation and consequent release of pro-inflammatory mediators. When the Aβ deposition process has already begun, NSAIDs may no longer be effective and may even be detrimental because of their inhibitory activity on chronically activated microglia that on long-term may mediate Aβ clearance.

CHF 5074 is an anti-inflammatory NSAID derivative in development for the treatment of the early stages of AD. It has a novel mechanism of action and other features that differentiate it from previously tested NSAIDs (Sivilia et al., BMC Neurosci. (2013) 14:44, which is incorporated herein by reference in its entirety). In particular, CHF 5074 is currently targeted for the treatment of individuals with mild cognitive impairment (MCI) due to AD who carry one or two apolipoprotein E ε4 alleles (APOE4 carriers). CHF 5074 is also being considered as a treatment for individuals at increased genetic risk of developing AD (APOE4 carriers with parental history of AD). The drug emerged from a discovery program that was aimed at obtaining aryl-propionic acid derivatives with Aβ42 lowering properties but devoid of COX inhibitory activity (Peretto et al., J. Med. Chem. (2005) 5705-5720, which is incorporated herein by reference in its entirety). CHF 5074 was selected from a chemical series of about 170 newly synthesized compounds for its selective Aβ42 inhibitory activity based on in vitro assays designed to measure a shift from Aβ42 to Aβ40, lack of effects on Notch processing and favorable pharmacokinetic profile (good oral absorption, satisfactory brain penetration, long half-life). However, subsequent tests conducted both in transgenic mouse models of AD and humans showed that CHF 5074 does not affect soluble concentrations of Aβ, indicating that plaque reduction occurs as the result of a gamma-secretase independent mechanism. In mouse mixed astrocytes-microglia culture, CHF 5074 has been shown to modulate microglial function by blunting or inhibiting M1 inflammatory activity and simultaneously stimulating M2 phagocytic responses to an Aβ42 stimulus (Lanzillotta et al., Conference on Alzheimer's Disease and Parkinson's Disease (2013) March 6-10, which is incorporated herein by reference in its entirety). In vivo, CHF5074 has been shown to inhibit brain plaque deposition and attenuate or reverse associated memory deficits in various human APP transgenic mice models of AD (Imbimbo et al., J. Pharmacol. Ther. (2007) 323: 822-830; Imbimbo et al., Br. J. Pharmacol. (2009) 156: 982-993; Imbimbo et al., J. Alzheimer's Dis. (2010) 20: 159-173; Balducci et al., J. Alzheimer's. Dis. (2011) 24:799-816; Lanzillotta et al., J. Mol. Neurosci. (2011) 45: 22-31; Guiliani et al., J. Neurochem. (2013) 124: 613-620; Silvia et al., BMC Neurosci. (2013) 14:44; Imbimbo et al., Alzheimer's Dis. Assoc. Disord (2013) 27:278-286; Ross et al., Curr. Alzheimer Res. (2013), all of which are incorporated herein by reference in their entireties).

Studies in healthy subjects (Imbimbo et al., Alzheimer's Dis. Assoc. Disord (2013) 27:278-286, which is incorporated herein by reference in its entirety) and in individuals with MCI (Ross et al., Curr. Alzheimer Res. (2013), which is incorporated herein by reference in its entirety) have shown that the drug lowers, in a dose-dependent fashion, CSF biomarkers of neuroinflammation, such as TNF-α and soluble CD 40 ligand (sCD40L), indicating a direct involvement of microglia.

Thus, there remains a need for formulations and methods for treating Alzheimer's disease and other proteinopathies by combination therapy. Therapeutic compositions and methods for therapeutic intervention in established proteinopathies or prior to their preclinical manifestation, as well as diagnostic agents and compositions for use in diagnosis and monitoring of proteinopathies may be of great value.

### SUMMARY OF THE INVENTION

Accordingly, it is one object of the present invention to provide novel improved therapeutic agents and methods for the treatment of proteinopathies.

It is another object of the present invention to provide novel methods of increasing the efficacy and decreasing the side effects associated with the therapeutic agents for the treatment of proteinopathies.

It is another object of the present invention to provide novel methods of modulating microglial phagocytic activity by administering a therapeutically effective amount of a 1-phenylalkanecarboxylic acid to facilitate microglial phagocytic activity and an effective amount(s) of one or more additional neuroprotective agent(s) to augment the effect of the 1-phenylalkanecarboxylic acid.

It is another object of the present invention to provide novel methods of modulating microglial phagocytic activity by administering a therapeutically effective amount of a 1-phenylalkanecarboxylic acid to prevent or slow down microglial inflammatory activity and an effective amount(s) of one or more additional neuroprotective agent(s) to augment the effect of the 1-phenylalkanecarboxylic acid.

It is another object of the present invention to provide novel pharmaceutical compositions comprising a 1-phenylalkanecarboxylic acid together with a neuroprotective agent in the prevention or therapeutic treatment of neurodegenerative diseases, in particular Alzheimer's disease, including slowing the progression or ameliorating symptoms of these diseases in either the preclinical or clinical stages of these diseases.

It is another object of the present invention to provide novel combination therapy for mammals, in particular humans, in the prevention or therapeutic treatment of proteinopathies and/or neurodegenerative diseases, including delaying the onset, slowing the progression or ameliorating symptoms of these diseases, comprising the administration of a therapeutically effective amount of 1-phenylalkanecarboxylic acid and a therapeutically effective amount at least one additional neuroprotective agent.

These and other objects, which will become apparent during the following detailed description, have been achieved by the inventors' discovery of a method of prevention or therapeutic treatment of proteinopathies and/or neurodegenerative diseases, including delaying the onset, slowing the progression or ameliorating symptoms of these diseases, comprising administering a 1-phenylalkanecarboxylic acid, a pro-drug of the 1-phenylalkanecarboxylic acid, a pharmaceutically acceptable salt or complex of any of the foregoing and at least one additional neuroprotective agent to a mammal, in particular a human, in need of such treatment. The neuroprotective agent(s) may be selected from the group consisting of β-amyloid peptides level reducers, pathogenic level tau reducers, microtubule stabilizers, agents capable or removing atherosclerotic plaques, agents that lower circulating levels of β-amyloid and tau, modulators of autophagy, neurotransmitter level regulators, GABA receptors antagonists, and additional agents that help maintain and/or restore cognitive function and functional deficits of AD, and/or slow down decline in cognitive functions and functional deficits in AD. The 1-phenylalkanecarboxylic acid and the additional neuroprotective agent(s) may be administered in the same or different compositions. The additional neuroprotective agent(s) may be administered before, concurrently with or after the administration of the 1-phenylalkanecarboxylic acid.

The present invention is also directed to the methods of decreasing neuroinflammation biomarkers in a mammal comprising administering a 1-phenylalkanecarboxylic acid, a pro-drug of the 1-phenylalkanecarboxylic acid, a pharmaceutically acceptable salt or complex of any of the foregoing and at least one additional neuroprotective agent selected from the group consisting of β-amyloid peptides level reducers, pathogenic level tau reducers, microtubule stabilizers, agents capable or removing atherosclerotic plaques, agents that lower circulating levels of β-amyloid and tau, modulators of autophagy, neurotransmitter level regulators, GABA receptors antagonists, and additional agents that help maintain and/or restore cognitive function and functional deficits of AD, and/or slow down decline in cognitive functions and functional deficits in AD in effective amounts to decrease neuroinflammation in the mammal. The additional neuroprotective agent(s) may be administered before, concurrently with or after the administration of the 1-phenylalkanecarboxylic acid.

The present invention is also directed to the methods of improving cognitive benefit in executive function and/or verbal memory in a mammal comprising administering a 1-phenylalkanecarboxylic acid, a pro-drug of the 1-phenylalkanecarboxylic acid, a pharmaceutically acceptable salt or complex of any of the foregoing and at least one additional neuroprotective agent selected from the group consisting of β-amyloid peptides level reducers, pathogenic level tau reducers, microtubule stabilizers, agents capable or removing atherosclerotic plaques, agents that lower circulating levels of β-amyloid and tau, modulators of autophagy, neurotransmitter level regulators, GABA receptors antagonists, and additional agents that help maintain and/or restore cognitive function and functional deficits of AD, and/or slow down decline in cognitive functions and functional deficits in AD in effective amounts to improve cognitive benefit in executive function and/or verbal memory in the mammal. The additional neuroprotective agent(s) may be administered before, concurrently with or after the administration of the 1-phenylalkanecarboxylic acid.

The present invention is further directed to pharmaceutical compositions comprising 1-phenylalkanecarboxylic acids, pro-drugs of 1-phenylalkanecarboxylic acids, bioesters on the carboxylic moiety of 1-phenylalkanecarboxylic acids, and pharmaceutically acceptable salts and complexes of any of the foregoing for use in the methods of the present invention.

The present invention is further directed to pharmaceutical compositions comprising 1-phenylalkanecarboxylic acids, pro-drugs of 1-phenylalkanecarboxylic acids, bioesters on the carboxylic moiety of 1-phenylalkanecarboxylic acids, and pharmaceutically acceptable salts and complexes of any of the foregoing, together with a neuroprotective agent selected from the group consisting of β-amyloid peptides level reducers, pathogenic level tau reducers, microtubule stabilizers, agents capable or removing atherosclerotic plaques, agents that lower circulating levels of β-amyloid and tau, modulators of autophagy, neurotransmitter levels regulators, GABA receptors antagonists and additional agents that help maintain and/or restore cognitive function and functional deficits of AD, and/or slow down decline in cognitive functions and functional deficits in AD, the process for the preparation thereof, and the use thereof in the prevention or therapeutical treatment of neurodegenerative diseases, in particular AD.

In certain aspects, an object of the present invention is to provide formulations containing a specified dose of CHF 5074 singly in the prevention, delaying onset or therapeutical treatment of proteinopathies and/or neurodegenerative diseases, in particular Alzheimer's disease, and for use in the methods of the present invention.

The present invention is also directed in part to a combination therapy for the treatment of one or more proteinopathies, including delaying the onset, slowing the progression or ameliorating symptoms of these diseases, comprising administering to a mammal (e.g., human patient) in need of such treatment a therapeutically effective dose of 1-phenylalkanecarboxylic acid, its pro-drug, bioisoster on the carboxylic moiety, or a pharmaceutically acceptable salt or complex of anyone of the foregoing together with a therapeutically effective amount(s) of one or more of the following: (1) β-amyloid peptides level reducers, (2) pathogenic level tau reducers, (3) microtubule stabilizers, (4) agents capable or removing atherosclerotic plaques, (5) agents that lower circulating levels of β-amyloid and tau, (6) modulators of autophagy, (7) neurotransmitter levels regulators, (8) GABA receptors antagonists, and (9) additional agents that help maintain and/or restore cognitive function and functional deficits of AD, and/or slow down decline in cognitive functions and functional deficits in AD.

The present invention is further directed in part to a combination therapy for the treatment of one or more proteinopathies, including delaying the onset, slowing the progression or ameliorating symptoms of these diseases, comprising administering to a mammal (e.g., human patient) in need of such treatment a therapeutically effective dose of 1-phenylalkanecarboxylic acids, their pro-drugs, and bioisosters on the carboxylic moiety, or a pharmaceutically acceptable salt or complex of anyone of the foregoing together with a therapeutically effective amount of one or more of the following: (1) an antibody capable of selectively recognizing a pathogenic conformation of soluble prefibrillar pathological or neurotoxic tau and its precursors; (2) an isolated immunogenic peptide comprising an epitope for an antibody capable of selectively recognizing a conformation of prefibrillar pathological or neurotoxic tau and its precursors; (3) an β-amyloid antibody that is end specific for a free N-terminus of the β-amyloid peptide or a free C-terminus of β-amyloid peptide; (4) an β-amyloid antibody that binds a mid-domain of the peptide but not full length APP; (5) a tau antibody that binds normal tau protein, (6) an isolated immunogenic peptide comprising an epitope for an antibody capable of selectively recognizing a free N-terminus of β-amyloid peptide or a free C-terminus of β-amyloid peptide, (7) an antibody that is specific for hTau40 truncated at its C-terminus at the glutamic acid residue Glu391, hTau40 truncated at the aspartic acid residue Asp421, hTau40 truncated at its N-terminus at the aspartic acid residue Asp13, proteins homologous to hTau40 truncated at its C-terminus at the glutamic acid residue Glu391, proteins homologous to hTau40 truncated at the aspartic acid residue Asp421, and proteins homologous to hTau40 truncated at its N-terminus at the aspartic acid residue Asp13, the antibody showing no binding and/or reactivity to a full length hTAu40, (8) an isolated immunogenic peptide comprising an epitope of an antibody that is specific for hTau40 truncated at its C-terminus at the glutamic acid residue Glu391, hTau40 truncated at the aspartic acid residue Asp421, hTau40 truncated at its N-terminus at the aspartic acid residue Asp13, proteins homologous to hTau40 truncated at its C-terminus at the glutamic acid residue Glu391, proteins homologous to hTau40 truncated at the aspartic acid residue Asp421, and proteins homologous to hTau40 truncated at its N-terminus at the aspartic acid residue Asp13, the antibody showing no binding and/or reactivity to a full length hTAu40, (9) a tau oligomeric complex-1 (TOC-1 or TEXAS Mab) monoclonal antibody, (10) an antibody comprising a variable region of the heavy chain which is the same or homologous as the heavy variable region of a tau oligomeric complex-1 (TOC-1 or TEXAS Mab) monoclonal antibody, (11) a conjugate of a cytoprotective agent (e.g., an antioxidant (e.g., melatonin or tocopherol) or an agent which will facilitate and/or improve antibody's ability to cross the blood brain barrier (BBB) (e.g., a hydrophobic substance which is capable of crossing the BBB, and is generally recognized as safe (GRAS) by the United States Food and Drug Administration (“FDA”) with one or more of any of the preceding antibodies. The therapeutically effective dose of 1-phenylalkanecarboxylic acids, their pro-drugs, and bioisosters on the carboxylic moiety and the therapeutically effective amount of one or more of the preceding agent may be administered in the same or different compositions. The combination therapy includes concurrent and sequential administration of 1-phenylalkanecarboxylic acids, their pro-drugs, and bioisosters on the carboxylic moiety and the preceding agents.

In certain aspects, the present invention is directed to pharmaceutical compositions (formulations) containing a specified dose of CHF 5074 singly or in combination with a drug that lowers β-amyloid peptide and/or reduces other pathological components in the disease administered as part of a combined treatment regimen.

In certain aspects, the present invention is further directed to an antibody-drug conjugate comprising CHF 5074 chemically linked to an amyloid-clearing antibody for use in the methods of the present invention.

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of a β-amyloid peptides level reducer to a mammal in need thereof, and pharmaceutical compositions for use in the combination therapy. The 1-phenylalkanecarboxylic acid may, e.g., be CHF 5074, a pharmaceutically acceptable salt or complex thereof, or a pro-drug thereof, and the β-amyloid peptides level reducer may, e.g., be selected from the group consisting of agents inhibiting synthesis of APP, agents that prevent formation of Aβ peptides, inhibitors of mGlu2/3 auto-receptor, alpha-secretase modulators, beta-secretase inhibitors, gamma-secretase inhibitors, gamma-secretase modulators, 5-HT4 agonists, antibodies to β-amyloid, immunogenic peptides that results in the production of antibodies to β-amyloid, blockers of oligomers' aggregation, fibril formation inhibitors, RAGE antagonists, and combinations of any two or more of the foregoing.

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of a pathogenic level tau reducer to a mammal in need thereof, and pharmaceutical compositions for use in the combination therapy. The 1-phenylalkanecarboxylic acid may, e.g., be CHF 5074, a pharmaceutically acceptable salt or complex thereof, or a pro-drug thereof, and the pathogenic level tau reducer may, e.g., be selected from the group consisting of tau formation inhibitors, antibodies to truncated tau, immunogenic peptides which result in the production of antibodies to truncated tau, tau phosphorylation blockers, tau aggregation inhibitors, and combinations of two or more the foregoing.

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of a microtubule stabilizer to a mammal in need thereof, and pharmaceutical compositions for use in the combination therapy. The 1-phenylalkanecarboxylic acid may, e.g., be CHF 5074, a pharmaceutically acceptable salt or complex thereof, or a pro-drug thereof, and the microtubule stabilizer may, e.g., be DBMS-241027 (Epothilone D).

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of an agent capable of removing atherosclerotic plaques to a mammal in need thereof, and pharmaceutical compositions for use in the combination therapy. The 1-phenylalkanecarboxylic acid may, e.g., be CHF 5074, a pharmaceutically acceptable salt or complex thereof, or a pro-drug thereof, and the agent capable of removing atherosclerotic plaques may, e.g., be a BET protein inhibitor.

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of an agent that lowers circulating levels of β-amyloid and tau to a mammal in need thereof. The 1-phenylalkanecarboxylic acid may, e.g., be CHF 5074, a pharmaceutically acceptable salt or complex thereof, or a pro-drug thereof, and the agent that lowers circulating levels of β-amyloid and tau may, e.g., be a nomethiazole (e.g., Sgc-1061).

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of a modulator of autophagy to a mammal in need thereof. The 1-phenylalkanecarboxylic acid may, e.g., be CHF 5074, a pharmaceutically acceptable salt or complex thereof, or a pro-drug thereof, and the modulator of autophagy may, e.g., be LNK-754, a peroxisome proliferator-activated receptor, an alpha/gamma agonist, an agent that reduce glucocorticoid activity, or combinations of two or more of any of the foregoing.

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of a neurotransmitter levels regulator to a mammal in need thereof. The 1-phenylalkanecarboxylic acid may, e.g., be CHF 5074, a pharmaceutically acceptable salt or complex thereof, or a pro-drug thereof, and the neurotransmitter levels regulator may, e.g., be selected from the group consisting of acetylcholinesterase inhibitors, butyrylcholinesterase inhibitors, MAO-B inhibitors, serotonin receptor antagonists, histamine receptor 3 (H3) antagonists, NMDA receptor antagonists, and combinations of two or more of the foregoing.

The present invention is further directed in part to a combination therapy comprising an administration of a therapeutically effective amount of a 1-phenylalkanecarboxylic acid (e.g., CHF 5074), its pro-drug, a bioisoster on the carboxylic moiety, or a pharmaceutically acceptable salt or complex of any of the foregoing together with a therapeutically effective amount of GABA receptors antagonists.

The present invention is further directed in part to a combination therapy comprising an administration of a therapeutically effective amount of a 1-phenylalkanecarboxylic acid, its pro-drug, a bioisoster on the carboxylic moiety, or a pharmaceutically acceptable salt or complex of any of the foregoing together with a therapeutically effective amount of an additional agent that help maintain and/or restore cognitive function and functional deficits of AD, and/or slow down decline in cognitive functions and functional deficits in AD to a mammal in need thereof. The 1-phenylalkanecarboxylic acid may, e.g., be CHF 5074, a pharmaceutically acceptable salt or complex thereof, or a pro-drug thereof, and the additional agent may, e.g., be selected from the group consisting of alpha-4 beta-2 nicotinic receptor modulators, M1 selective muscarinic agonists, Alpha4/beta2 neuronal nicotinic receptor agonists, α-7 nicotinic acetylcholine receptor (α7-nAChR) allosteric modulators, insulin sensitizers, calpain inhibitors, neurotrophic agents, nicotinic receptor agonists and combinations of two or more of any of the foregoing.

The present invention is further directed in part to combination therapy via the administration of pharmaceutical compositions comprising 1-phenylalkanecarboxylic acids together with, e.g., isolated antibodies (e.g., non-naturally occurring antibodies or genetically engineered antibodies) capable of selectively recognizing prefibrillar pathological or neurotoxic tau, including their pathogenic conformations. These antibodies may reduce or eliminate toxicity of the pathological tau and its precursors and/or slow down or prevent aggregation of the pathological tau into insoluble filaments. These antibodies may also lower the amount of pathogenic tau and its precursors in the brain and CSF fluid of a mammal, and may delay or prevent memory decline and other symptoms of tauopathies, including symptoms of AD, in the mammal. Because these antibodies are selective for the pathological tau and its precursors, these antibodies are not expected to affect biological functions of normal tau in vivo. In the preferred embodiments, the antibody has an equilibrium constant KD with the antigen for which it is selective of from 1×10⁻⁹ M to 1×10⁻¹¹ M in-vitro; and has an equilibrium constant KD with other peptides or proteins (e.g., htau40) which is from 1×10⁻⁴M to 1×10⁻⁶ M or shows no detectible binding or reactivity with these other peptides or proteins in-vitro, when tested at the saturating level of antibody-immunogen binding using 0.1 μg/ml of the antibody on a dot blot with 50 ng of the peptide or protein. These antibodies may also allow for early treatment of tauopathies (e.g., AD), e.g., at least 10 years before signs of cognitive decline or dementia appear and before NFTs begin to form, because these antibodies selectively recognize neurotoxic tau or its pathogenic conformations which begin to appear in mammals suffering from or at risk of developing a tauopathy (e.g., AD) at least 10 years before symptoms of dementia begin to appear.

The present invention is also directed in part to combination therapy via the administration of pharmaceutical compositions comprising 1-phenylalkanecarboxylic acids together with an isolated immunogenic peptide (e.g., a genetically engineered peptide) comprising an epitope of an antibody capable of selectively recognizing prefibrillar pathological or neurotoxic tau and precursors thereof, including their pathogenic conformations. The immunogenic peptide of the invention is capable of inducing production of the antibodies (e.g., i.e., non-naturally occurring antibodies or genetically engineered antibodies) capable of selectively recognizing the prefibrillar pathological or neurotoxic tau and precursors thereof in a mammal, upon administration to the mammal. These antibodies may be used for therapeutic intervention in and/or prevention of tauopathies (e.g., AD). This method encompasses both in situ and ex situ production of the antibodies capable of selectively recognizing prefibrillar pathological or neurotoxic tau and its precursors, including their pathogenic conformations.

The present invention is further directed to combination therapy via the administration of pharmaceutical compositions comprising 1-phenylalkanecarboxylic acids together with one or more antibodies (e.g., i.e., non-naturally occurring antibodies or genetically engineered antibodies) capable of selectively recognizing prefibrillar pathological or neurotoxic tau and precursors thereof, including their pathogenic conformations, and pharmaceutical compositions comprising immunogenic peptides comprising epitopes of the antibodies capable of selectively recognizing prefibrillar pathological or neurotoxic tau and precursors thereof, including their pathogenic conformations. These compositions may be used for therapeutic intervention in and/or prevention of tauopathies, including AD.

In another aspect, the present invention is directed to combination therapy via the administration of pharmaceutical compositions comprising a 1-phenylalkanecarboxylic acid together with a vaccine comprising the antibodies capable of selectively recognizing the prefibrillar pathological or neurotoxic tau and precursors thereof, including their pathogenic conformations, or/and the immunogenic peptides comprising epitopes of the antibodies capable of selectively recognizing prefibrillar pathological or neurotoxic tau and precursors thereof. The vaccine may include one or more additional active agents (e.g., antibodies and/or immunogens) for the treatment or prevention of tauopathies, including AD.

The present invention is also directed to combination therapy via the administration of pharmaceutical compositions comprising a 1-phenylalkanecarboxylic acid together with the administration to a subject in need of therapy for a tauopathy (e.g., AD) of a therapeutically effective dose of the antibodies capable of selectively recognizing prefibrillar pathological or neurotoxic tau and precursors thereof, and/or their pathogenic conformations, or/and of the immunogenic peptides comprising epitopes of the antibodies capable of selectively recognizing prefibrillar pathological or neurotoxic tau and precursors thereof. Administration of these active agents is expected, e.g., to delay or reduce tau pathology in mammals suffering from or at risk of developing a tauopathy and/or improve cognitive function in these mammals. Administration of these active agents is also expected to neutralize and/or promote clearance of the pathological tau and its precursors, reduce or eliminate toxicity of the pathological tau and its precursors and/or slow down or prevent aggregation of the pathological tau into insoluble filaments, all without affecting the biological functions of normal tau. Thus, administration of these agents is expected to delay or prevent memory decline and other symptoms of tauopathies, including symptoms of AD in these mammals.

The present invention is also related to a combination therapy via the administration of pharmaceutical compositions comprising 1-phenylalkanecarboxylic acids together with an immunization of a mammal comprising administering a therapeutically effective dose of the antibodies capable of selectively recognizing prefibrillar pathological or neurotoxic tau and precursors thereof, including their pathological conformations, or/and of immunogenic peptides comprising epitopes of antibodies capable of selectively recognizing prefibrillar pathological or neurotoxic tau and precursors thereof, to the mammal.

The present invention is also directed to methods for the prevention or therapeutical treatment of proteinopathies and/or neurodegenerative diseases combination therapy via the administration of pharmaceutical compositions comprising 1-phenylalkanecarboxylic acids together with inducing an immunologic response in a mammal comprising administering a therapeutically effective dose of an immunogenic peptide(s) comprising epitope(s) of the antibodies capable of selectively recognizing prefibrillar pathological or neurotoxic tau and precursors thereof to the mammal.

The present invention is additionally directed to combination therapy via the administration of pharmaceutical compositions comprising 1-phenylalkanecarboxylic acids together with a pharmaceutical composition(s) which include a (e.g., recombinant) antibody that discriminates between a β-amyloid peptide and the β-amyloid protein precursor (APP) from which it is proteolytically derived. Preferably, these antibodies are end-specific anti-β-amyloid antibodies which are generated, e.g., from an immunogenic peptide incorporating either a free N-terminus or a free C-terminus of a β-amyloid peptide involved in the pathogenesis of Alzheimer's disease.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 provides a graph plotting the week 88 (end of the open-label extension of the Phase 2 study in MCI patients) change from baseline for verbal memory. As it can be ascertained from the data, the results obtained with the 200 mg/day and the 400 mg/day dosages were surprisingly superior with respect to verbal memory as compared to the results obtained with the 600 mg/day dose.

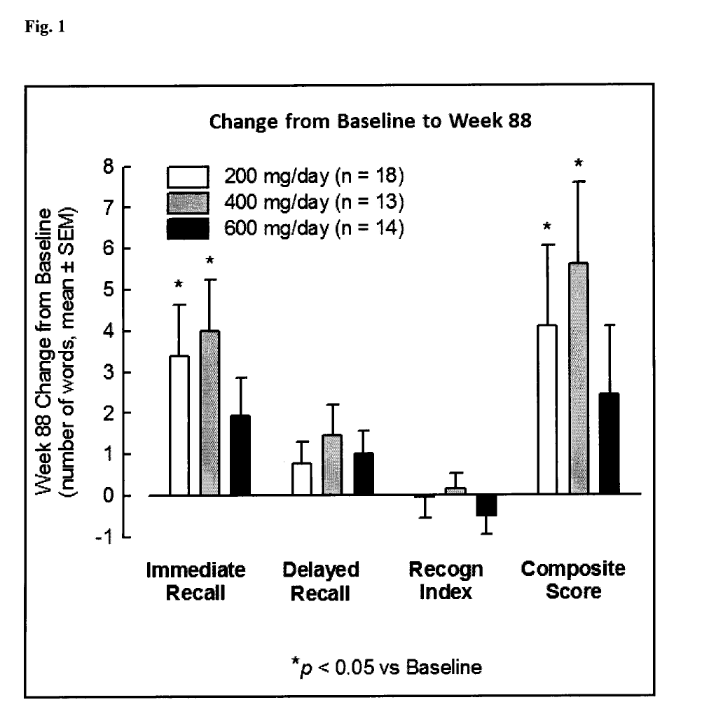

FIG. 2 is a graph which depicts the level of CHF 5074 in cerebrospinal fluid (CSF) for the 200 mg/day, the 400 mg/day and the 600 mg/day doses. The results depicted in FIG. 2 were taken at Day 85 of the Study.

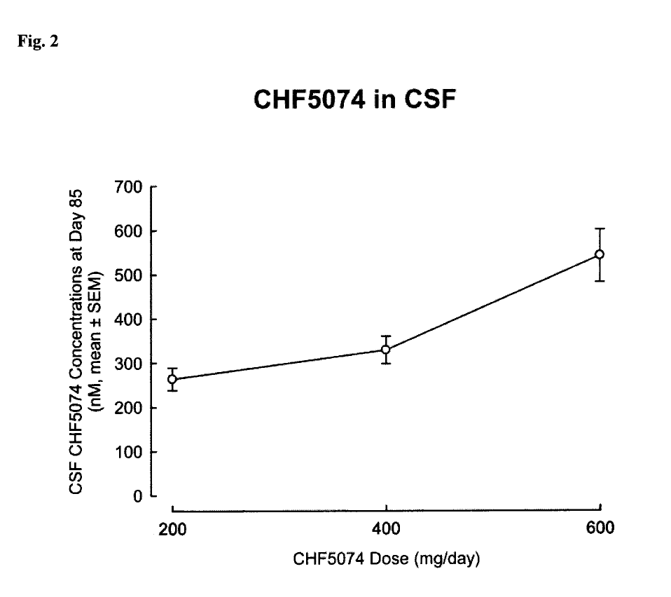

FIG. 3 is a graph showing the level of TNF-α in CSF obtained with each of the administered doses (the 200 mg/day, the 400 mg/day and the 600 mg/day doses).

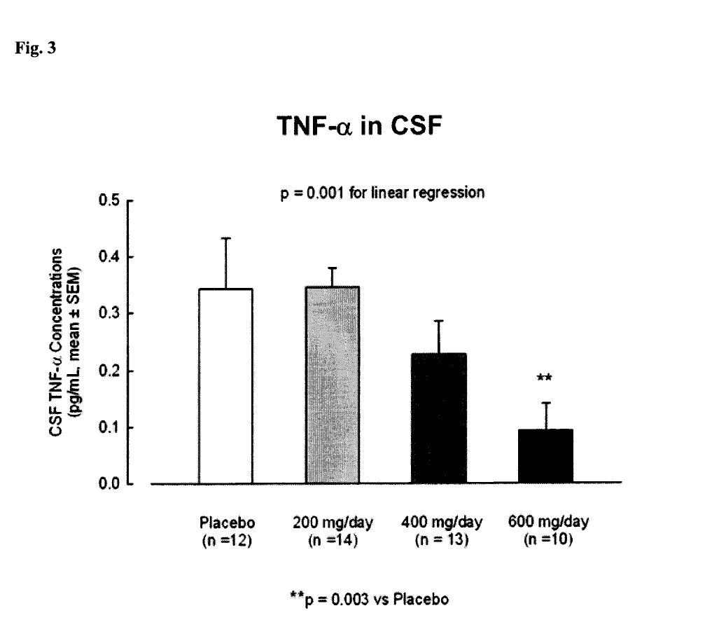

FIG. 4 is Table providing the results of cognitive tests at weeks 52 and 88 in the Study.

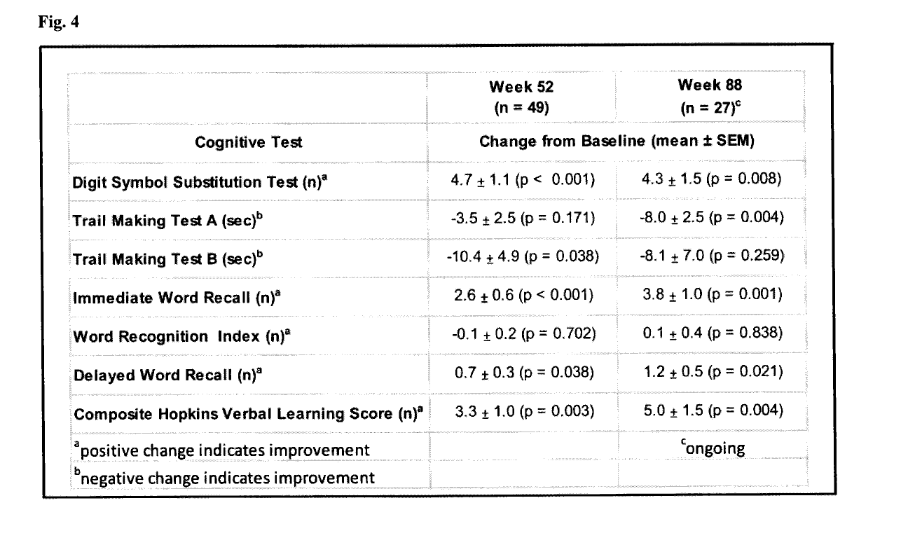

FIG. 5 is a graph depicting the effects of prolongs treatment with CHF 5074 on verbal memory (immediate word recall).

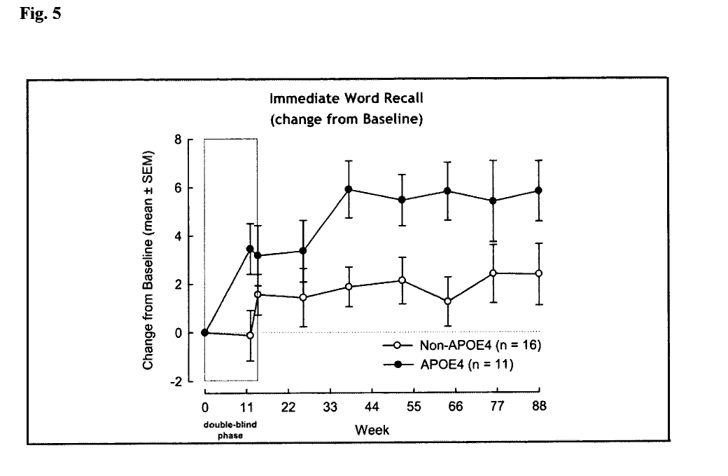

FIG. 6 is a graph depicting the effects of prolonged treatment with CHF 5074 on verbal memory (delayed word recall).

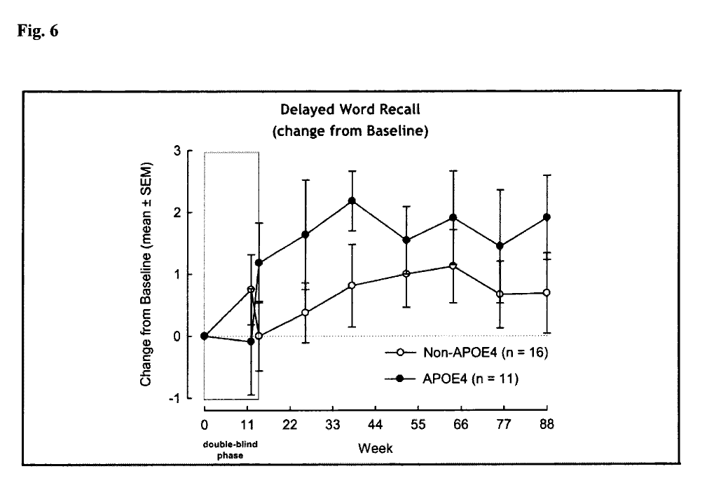

FIG. 7 is a graph depicting the effects of prolongs treatment with CHF 5074 on verbal memory (Total Hopkins Verbal learning Score).

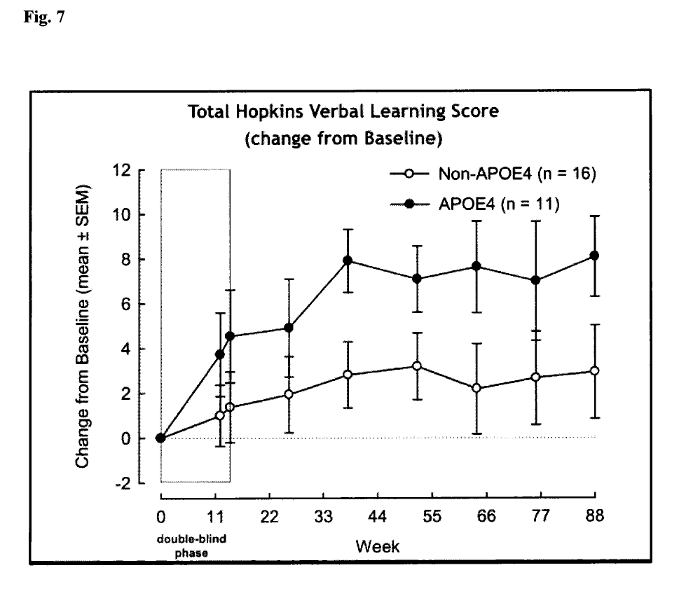

FIG. 8 is a graph showing the dose-dependent improvement in verbal memory at week 88 of the Study (number of words, mean±SEM) for the 200 mg/day and the 400 mg/day doses.

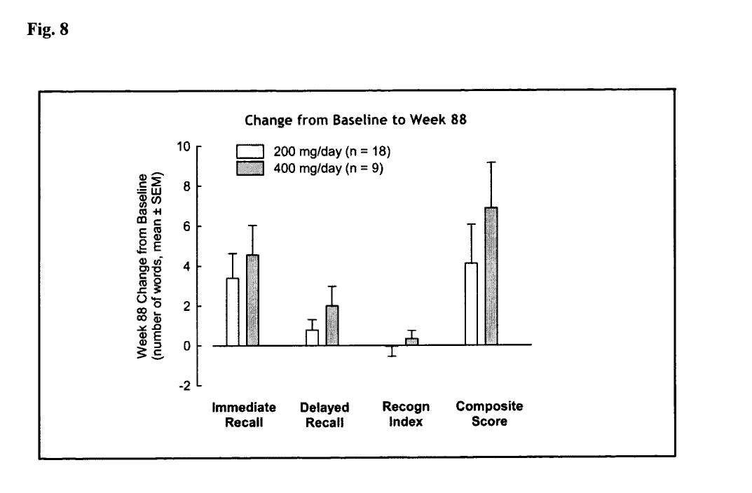

FIG. 9 is a graph showing the effects of prolonged treatment with CHF 5074 on executive function in the Study (Trail Making Test A).

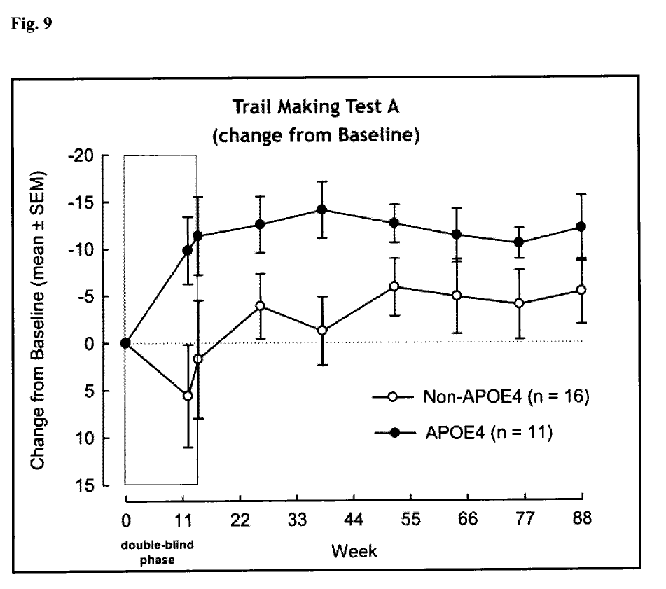

### DETAILED DESCRIPTION OF THE PREFERRED EMBODIMENTS

The term “antibody” as used in the present application includes whole antibodies and binding fragments/segments thereof.

The terms “does not bind,” “does not recognize,” and “does not show reactivity” as used in the present application mean either that an antibody show no detectible binding or reactivity with a peptide or protein (e.g., hTau40 or its recombinant form) in-vitro, defined as having an equilibrium constant KD with the peptide or protein of from 1×10⁻⁴M to 1×10⁻⁶ M, and as determined for example when tested at the saturating level of antibody-immunogen binding using 0.1 μg/ml of the antibody on a dot blot with 50 ng of the peptide or protein.

The terms “binds selectively,” “selectively recognize,” “selectively recognizes,” “selectively recognizing,” “having selectivity,” and “selective for” as used in the present specification mean that an antibody is at least seven times more likely to bind the antigen it is selective for than other proteins or peptides, when tested using immunogold labeling using 0.4 μg/ml of the purified antibody.

The term “conformation” means a three-dimensional form of a peptide or protein (e.g., a secondary structure of the peptide or protein).

“Conformation selective antibody” as used in the present specification means that the antibody is selective for the specific conformation (e.g., secondary structure of the antigen). A conformation selective antibody would not recognize the amino acid sequence of its antigen when that sequence is not in the conformation selectively recognized by the antibody, when tested at the saturating level of antibody-immunogen binding using 0.1 μg/ml of the antibody on a dot blot with 50 ng of antigen.

The term “filament(s)” refers to structure(s) of tau aggregates which is (are) greater than 50 nm in length.

The term “human antibody” in the present application includes antibodies having variable and constant regions derived from human immunoglobulin sequences. The term “human antibody,” as used in the present application, does not include antibodies in which CDR sequences from another mammalian species, e.g., a mouse, have been grafted onto human framework sequences.

The term “humanized antibody” as used in the present application refers to antibodies which comprise heavy and light chain variable region sequences from a non-human species (e.g., a mouse) but in which at least a portion of the V_{H} and/or V_{L} sequence has been replaced with a corresponding portion from a human immunoglobulin sequence.

The term “neuroprotective agent” as used in the present application refers to any agent which can prevent, attenuate or treat proteinopathies and/or neurodegenerative diseases, in particular Alzheimer's disease. The term “neuroprotective agent” is intended to encompass, but not be limited to, agents, antibodies, vaccines or medicines known to those having ordinary skill in the art such as an β-amyloid antibody or a neurotoxic tau antibody; a gene therapy for the treatment of a proteinopathy; a vaccine for β-amyloid antibody or a neurotoxic tau, a neurotransmitter receptor modulator; an alpha-4 beta-2 nicotinic receptor modulator; a soluble amyloid reducing/clearing agent; a serotonin 6 receptor antagonist, a histamine-3 receptor antagonist, a β-secretase inhibitor, a β-amyloid protein inhibitor, a microtubule stabilizer, a gamma-secretase modulator, a BACE1 protein inhibitor, an α7-nAChR agonist, a 5-HT6 antagonist, an immune globulin, a MAO-B inhibitor, a BET protein inhibitor, a H3 antagonist, 5-HT4 agonist, a RAGE antagonist, a conjugate of melatonin, and mixtures of any of the foregoing.

The term “oligomer(s)” as used in the present application refers to tau aggregates which are less than 50 nm in length and which are intermediates between monomers of Tau and NFTs. The term “oligomer(s)” does not include monomers of tau (e.g., hTau40), dimers of tau and NFTs.

The terms “tau protein” and “tau monomer” as used in the present application refer to any one of known isoforms of tau (e.g., hTau40, the longest isoform of human microtubule associated protein tau containing all alternatively spliced inserts).

The term “immunogen” refers to a molecule capable of being bound by an antibody, a B cell receptor (BCR), or a T cell receptor (TCR) if presented by MHC molecules. The term “immunogen,” as used herein, also encompasses T-cell epitopes. An immunogen can additionally be capable of being recognized by the immune system and/or being capable of inducing a humoral immune response and/or cellular immune response leading to the activation of B- and/or T-lymphocytes. This may, however, require that, at least in certain cases, the immunogen contains or is linked to a T helper cell epitope and is given an adjuvant. An immunogen can have one or more epitopes (e.g., B- and T-epitopes). The “immunogen” as used herein may also be mixtures of several individual immunogens. The term “immunogen” encompasses, but is not limited to an isolated immunogenic peptide.

The term “prefibrillar pathological or neurotoxic tau” includes pathological or neurotoxic tau oligomers and dimmers.

The term “substantially” in the context of antibody recognition means that any binding of the antibody to its antigen that may be exhibited is insufficient to affect normal functions of the antigen in vivo.

The term “tauopathy” refers to tau-related disorders or conditions, e.g., Alzheimer's Disease, Progressive Supranuclear Palsy (PSP), Corticobasal Degeneration (CBD), Pick's Disease, Frontotemporal dementia and Parkinsonism associated with chromosome 17 (FTDP-17), Parkinson's disease, stroke, traumatic brain injury, mild cognitive impairment and the like.

The abbreviation “AA” means “arachidonic acid.”

The abbreviation “Aβ” means “amyloid β.”

The abbreviation “AD” means “Alzheimer disease.”

The present invention is directed to the treatment of proteinopathies which include neurodegenerative diseases such as Alzheimer's disease (AD), Parkinson's disease (PD), Huntington's disease (HD), amyotrophic lateral sclerosis (ALS), Familial Amyloid Polyneuropathy (FAP), prion disease, inclusion body myositis and various forms of retinal degeneration such as age related macular degeneration (AMD).

In certain preferred embodiments, the invention is directed to the treatment of proteinopathies via combination therapy utilizing treatment of a mammal (e.g., human) in need of such treatment with 1-phenylalkanecarboxylic acids, their pro-drugs, and bioisosters on the carboxylic moiety together with one or more additional neuroprotective agents, e.g., a drug or an antibody that lowers β-amyloid and/or neurotoxic tau or its pathogenic conformations and/or reduces other pathological components in the disease. The 1-phenylalkanecarboxylic acids have been reported to have more selective and more potent inhibitory activity on the peptide β-amyloid₁₋₄₂ peptide while inhibiting to a lesser extent, or not inhibiting at all, cyclooxygenase would be a significant improvement in therapies aimed at preventing the onset of Alzheimer's disease and/or at delaying the cognitive decline that represents an early stage disease.

### 1-Phenylalkanecaroxylic Acids

In preferred embodiments, the 1-phenylalkanecarboxylic acids used in the pharmaceutical compositions of the invention has of general formula (I):

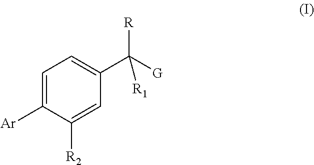

wherein:

R and R₁ are the same and are selected from the group of linear or branched C₁-C₄ alkyl; otherwise they form a 3 to 6 carbon atoms ring with the carbon atom to which they are linked;

G is: a COOR″ group wherein R″ is H, linear or branched C₁-C₄ alkyl, C₃-C₆ cycloalkyl or ascorbyl; a CONH₂ or a CONHSO₂R′″ group wherein R′″ is linear or branched C₁-C₄ alkyl or C₃-C₆ cycloalkyl; a tetrazolyl residue;

R₂ is H, CF₃, OCF₃ or a halogen selected from the group of F, Cl, Br, I, preferably fluorine;

Ar is a group of formula

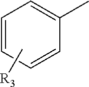

wherein R₃ represents one or more groups independently selected from: halogen as previously defined; CF₃; C₃-C₈ cycloalkyl optionally substituted with one or more C₁-C₄ alkyl and/or oxo groups; CH═CH₂; CN; CH₂OH; methylenedioxy or ethylenedioxy; NO₂; phenyl optionally substituted with one or more of the following groups: halogen; CF₃; OCF₃; OH; linear or branched C₁-C₄ alkyl; a saturated heterocycle with at least 4 carbon atoms and at least 1 heteroatom; C₃-C₈ cycloalkyl in turn optionally substituted with one or more of the following groups linear or branched C₁-C₄ alkyl, CF₃ or OH; OR₄ or NHCOR₄ wherein R₄ is CF₃, linear or branched C₂-C₆ alkenyl or alkynyl; benzyl; phenyl optionally substituted with one or more of the following groups: halogen, CF₃, OCF₃, OH, linear or branched C₁-C₄ alkyl; a saturated heterocycle with at least 4 carbon atoms and at least 1 heteroatom; C₃-C₈ cycloalkyl in turn optionally substituted with one or more of the following groups: linear or branched C₁-C₄ alkyl, CF₃ or OH; SR₅, SO₂R₅ or COR₅ wherein R₅ is linear or branched C₁-C₆ alkyl; otherwise Ar is an heterocycle ring selected from the group consisting of thiophene, benzothiophene, dibenzothiophene, thianthrene, pyrrole, pyrazole, furan, benzofuran, dibenzofuran, indole, isoindole, imidazole, benzoimidazole, oxazole, isoxazole, benzoxazole, thiazole, pyridine, pyrimidine, pyrazine, pyridazine, quinoline, isoquinoline, quinazoline, quinoxaline, cinnoline, pyrazole, pyran, benzopyran, pyrrolizine, phthalazine, 1,5-naphthyridine, 1,3-dioxole, 1,3-benzodioxole, optionally substituted with one or more groups R₃ as defined above;

and pharmaceutically acceptable salts and esters thereof.

A first group of preferred compounds is that in which: R and R₁ form a 3 carbon atoms ring with the carbon atom to which they are linked;

R₂ is fluorine;

G is COOR″, wherein R″ is H, linear or branched C₁-C₄ alkyl, C₃-C₆ cycloalkyl or ascorbyl;

Ar is phenyl as defined above.

A second group of preferred compounds is that in which:

R and R₁ form a 3 carbon atoms ring with the carbon atom to which they are linked; R₂ is fluorine; G is CONH₂ or CONHSO₂R′″ wherein R′″ is linear or branched C₁-C₄ alkyl or C₃-C₆ cycloalkyl; Ar is phenyl as defined above.

A third group of preferred compounds is that in which: both R and R₁ are methyl; R₂ is fluorine; G is COOR″ wherein R″ is as defined above; Ar is phenyl as defined above.

A fourth group of preferred compounds is that in which: both R and R₁ are methyl; R₂ is fluorine; G is CONH₂ or CONHSO₂R′″, wherein R′″ is as defined above; Ar is phenyl as defined above.

A fifth group of preferred compounds is that in which: R and R₁ form a 3 carbon atoms ring with the carbon atom to which they are linked; R₂ is fluorine; G is COOR″ wherein R″ is as defined above; Ar is a heterocycle as defined above.

A sixth group of preferred compounds is that in which: both R and R₁ are methyl; R₂ is fluorine; G is COOR″ wherein R″ is as defined above; Ar is a heterocycle as defined above.

The above compounds are further described in U.S. Pat. No. 7,662,995 (which is incorporated herein by reference in its entirety), filed on Oct. 10, 2006, which was a 371 of International Patent Application No. PCT/EP04/01596, filed on Feb. 19, 2004, and claims priority to Italian Patent Application No. MI2003A000311, filed on Feb. 21, 2003, and Italian Patent Application No. MI2003A002068, filed on Oct. 23, 2003.

In certain embodiments, derivatives of 1-phenylalkanecarboxylic acids wherein the carboxylic group is linked to a residue allowing the passage of the blood-brain barrier and the distribution of the active moiety in the brain are used in the formulations of the present invention. In an embodiment of the invention, said residue is represented by the amide of an alpha-amino acid and preferably is glycinamide.

Particularly preferred are the following compounds: 2-methyl-2(2-fluoro-4′-trifluoromethylbiphen-4-yl)propionic acid (CHF 4810); 2-methyl-2(2-fluoro-4′cyclohexyl biphen-4-yl)propionic acid (CHF 4961); 1-(2-fluoro-4′-trifluoromethylbiphenyl-4-yl)cyclopropanecarboxylic acid (CHF 5022); 1-(4′-cyclohexyl-2-fluorobiphenyl-4-yl)cyclopropanecarboxylic acid (CHF 5023); 1-(4′-benzyloxy-2-fluorobiphenyl-4-yl)cyclopropanecarboxylic acid (CHF 5042); 1-(2-fluoro-4′-isopropyloxybiphenyl-4-yl)cyclopropanecarboxylic acid (CHF 5044); 1-(2-fluoro-3′-trifluoromethoxybiphenyl-4-yl)cyclopropanecarboxylic acid (CHF 5045); 1-(2-fluoro-4′-trifluoromethoxybiphenyl-4-yl)cyclopropanecarboxylic acid (CHF 5046); 1-(2-fluoro-3′-trifluoromethylbiphenyl-4-yl)cyclopropanecarboxylic acid (CHF 5058); 1-(4′-cyclopentyl-2-fluorobiphenyl-4-yl)cyclopropanecarboxylic acid (CHF 5059); 1-(4′-cycloheptyl-2-fluorobiphenyl-4-yl)cyclopropanecarboxylic acid (CHF 5060); 1-(2′-cyclohexyl-2-fluorobiphenyl-4-yl)cyclopropanecarboxylic acid (CHF 5061); 1-(2-fluoro-4′-hydroxybiphenyl-4-yl)cyclopropanecarboxylic acid (CHF 5070); 1-[2-fluoro-4′-(tetrahydropyran-4-yloxy)biphenyl-4-yl]-cyclopropanecarboxylic acid (CHF 5071); 1-(2,3′,4′-trifluorobiphenyl-4-yl)cyclopropanecarboxylic acid (CHF 5073); 1-(3′,4′-dichloro-2-fluorobiphenyl-4-yl)cyclopropanecarboxylic acid (CHF 5074); 1-(3′,5′-dichloro-2-fluorobiphenyl-4-yl)cyclopropanecarboxylic acid (CHF 5075); 1-(3′-chloro-2,4′-difluorobiphenyl-4-yl)cyclopropanecarboxylic acid (CHF 5076); 1-(4-benzo[b]thiophen-3-yl-3-fluorophenyl)cyclopropanecarboxylic acid (CHF 5077); 1-(2-fluoro-4′-prop-2-inyloxy-biphenyl-4-yl)-cyclopropanecarboxylic acid (CHF 5078); 1-(4′-cyclohexyloxy-2-fluoro-biphenyl-4-yl)-cyclopropanecarboxylic acid (CHF 5079); 1-[2-fluoro-4′-(tetrahydropyran-4-yl)-biphenyl-4-yl]-cyclopropanecarboxylic acid (CHF 5080); 1-[2-fluoro-4′-(4-oxo-cyclohexyl)-biphenyl-4-yl]-cyclopropanecarboxylic acid (CHF 5081); 2-(2″-fluoro-4-hydroxy-[1,1′:4′,1″]tert-phenyl-4″-yl)-cyclopropanecarboxylic acid (CHF 5083); 1-[4′-(4,4-dimethylcyclohexyl)-2-fluoro[1,1′-biphenyl]-4-yl]-cyclopropanecarboxylic acid (CHF 5084); 1-[2-fluoro-4′-[[4-(trifluoromethyl)benzoyl]amino][1,1′-biphenyl]-4-yl]-cyclopropanecarboxylic acid (CHF 5094); 1-[2-fluoro-4′-[[4-(trifluoromethyl)cyclohexyl]oxy][1,1′-biphenyl]-4-yl]-cyclopropanecarboxylic acid (CHF 5096); 1-[2-fluoro-4′-[(3,3,5,5-tetramethylcyclohexyl)oxy][1,1′-biphenyl]-4-yl]-cyclopropanecarboxylic acid (CHF 5102); 1-[4′-[(4,4-dimethylcyclohexyl)oxy]-2-fluoro[1,1′-biphenyl]-4-yl]-cyclopropanecarboxylic acid (CHF 5103); 1-(2,3′,4″-trifluoro[1,1′:4′,1″-tert-phenyl]-4-yl)-cyclopropanecarboxylic acid (CHF 5104); 1-(2,2′,4″-trifluoro[1,1′:4′,1″-tert-phenyl]-4-yl)-cyclopropanecarboxylic acid (CHF 5105); 1-(2,3′-difluoro-4″-hydroxy[1,1′:4′,1″-tert-phenyl]-4-yl)-cyclopropanecarboxylic acid (CHF 5106); 1-(2,2′-difluoro-4″-hydroxy[1,1′:4′,1″-tert-phenyl]-4-yl)-cyclopropanecarboxylic acid (CHF 5107); and 2-(2-fluoro-3′,5′-bis(chloro)biphen-4-yl)propionic acid amide (CHF 5125). A more preferred group of compounds is that in which R and R₁ form a 3 carbon atoms ring with the carbon atom to which they are linked; R₂ is fluorine; G is COOH; Ar is phenyl substituted with one or more groups in such a way as that the log P (the partition coefficient between n-octanol and water) of the whole molecule is equal or higher than 4.5 as calculated in silico by using the software QikProp® release version 2.1 (Schrodinger Inc).

It has indeed been found that the higher the log P of the molecule, the greater is the inhibition potency of the release of Aβ42 peptide and that particularly potent compounds are those whose log P is equal or higher than 4.5, preferably higher than 5.0.

Examples of these compounds are CHF 5022, CHF 5074, CHF 5096, CHF 5105, CHF 5106 and CHF 5107.

In a most preferred embodiment, the 1-phenylalkanecarboxylic acid used in the pharmaceutical composition of the invention is CHF 5074.

CHF 5074 is a new microglial modulator that has been shown to prevent brain plaque deposition and attenuate memory deficits in transgenic mouse models of AD. As demonstrated in the appended examples, CHF 5074 dose-dependently lowers cerebrospinal fluid levels of two biomarkers of neuroinflammation (sCD40L and TNF-α).

The invention also relates to the pharmaceutically acceptable salts and esters prepared in order to increase the crossing of the blood brain barrier.

1-phenylalkanecarboxylic acids (CHF 5074) may decrease side effects associated with neuroprotective agents (e.g., β-amyloid peptides level reducers) and/or may potentiate the actions and increase efficacy of the neuroprotective agents.

### Neuroprotective Agents

A neuroprotective agent used in the compositions and methods of the present invention may be selected from the group consisting of β-amyloid peptides level reducers, pathogenic level tau reducers, microtubule stabilizers, agents capable or removing atherosclerotic plaques, agents that lower circulating levels of β-amyloid and tau, modulators of autophagy, neurotransmitter level regulators, GABA receptors antagonists, and additional agents that help maintain and/or restore cognitive function and functional deficits of AD, and/or slow down decline in cognitive functions and functional deficits in AD. The neuroprotective agent may selectively modulate microglial activity and/or potentiate efficacy of 1-phenylalkanecarboxylic acids used in the methods of the present invention.

β-Amyloid Peptides Levels Reducers

β-amyloid peptides level reducers inhibit formation of β-amyloid peptides, slow down and prevent aggregation/deposition of β-amyloid peptides, and/or facilitate removal of β-amyloid peptides.

β-Amyloid peptides level reducers may also reduce microglial load and/or facilitate microglial phagocytic activity, and/or prevent or slow down microglial inflammatory activity. β-Amyloid peptides level reducers may therefore potentiate the actions of 1-phenylalkanecaroxylic acids (e.g., CHF 5074).

A β-amyloid peptides level reducer may, e.g., be selected from the group consisting of agents inhibiting synthesis of APP, agents that prevent formation of β-amyloid peptides, inhibitors of mGlu2/3 auto-receptor, alpha-secretase modulators, beta-secretase inhibitors, gamma-secretase inhibitors, gamma-secretase modulators, 5-HT4 agonists, antibodies to β-amyloid peptides, immunogenic peptides that result in the production of antibodies to β-amyloid, blockers of oligomers' aggregation, fibril formation inhibitors, RAGE antagonists, and combinations of any two or more of the foregoing.

### Agents Inhibiting Synthesis of APP

Agents that inhibit synthesis of APP reduce the amount of APP available for degradation to β-amyloid peptides, and therefore reduce the amount of β-amyloid peptides and decrease microglial load.

Agents inhibiting synthesis of APP include, e.g., R-phenserine.

Agents that Prevent Formation of Aβ Peptides

Agents that prevent formation of Aβ peptides reduce the amount of Aβ peptides. These agents may, e.g., induce cleavage of APP into peptides different than pathogenic peptides.

Agents that prevent formation of β-amyloid include, e.g., azaindolizinone derivatives (e.g., ST101). ST101 induces APP cleavage such that a 17 kDa C-terminal fragments are produced, rather than Aβ peptides.

Inhibitors of mGlu2/3 Auto-Receptor

Activation of metabotropic glutamate receptor subtype 2 (mGluR2; GRM2) and/or mGluR3 (GRM3) by glutamate causes conversion of (APP) into β-amyloid (Aβ). Inhibition of mGlu2/3 auto-receptor should therefore reduce levels of Aβ and decrease microglial load.

Inhibitors of mGlu2/3 auto-receptor also stimulate serotonin release and, after chronic dosing, hippocampal neurogenesis.

Inhibitors of mGlu2/3 auto-receptor include, e.g., BCI-632, BCI-638 (an oral prodrug of BCI-632).

### Alpha-Secretase Modulators

Alpha-secretase cleaves APP into a soluble form, s-APPalpha, which is readily cleared from the brain. Alpha-secretase modulators should therefore lower levels of AP and decrease microglial load.

Alpha-secretase inhibitors include, e.g., APH-0703.

### Beta-Secretase Inhibitors (BACE1 Inhibitors)

Beta-secretase cleaves APP to form Aβ peptides.

Beta-secretase inhibitors (BACE1 inhibitors) decrease the production of AP peptides and may lower microglial load.

Beta-secretase inhibitors include, but are not limited to, BAN 2203, BAN2401, CTS-21166, E2609, MK-8931, E2609, and HPP-854.

### Metal-Protein Interaction-Attenuating Compounds

Metal-protein interaction-attenuating compounds (MPACs) reduce amyloid aggregation by interfering with the interaction of copper and zinc with beta amyloid

MPACs include, but are not limited to, the compound quoted as PBT2 and clioquinol.

### Gamma-Secretase Inhibitors

Gamma-secretase cleaves APP to form Aβ peptides.

Gamma-secretase inhibitors decrease the production of Aβ peptides and may lower microglial load.

Gamma-secretase inhibitors include, e.g., BMS-708163 (avagacestat) and ELND0005.

### Gamma-Secretase Modulators

Gamma-secretase modulators modify the relative proportions of the Aβ isoforms produced without changing the rate at which APP is processed.

Thus, gamma-secretase modulators decrease levels of Aβ peptides and may lower microglial load.

Gamma-secretase modulators include, e.g., BMS-932481, E-2212; E-2012, JNJ-40418677, GSM1, SPI-1802, SPI-1810, NIC5-15, and EVP-0962

### 5-HT4 Agonists

5-HT4 agonists increase the secretion of the non-amyloidogenic soluble amyloid precursor protein-alpha (sAPPalpha), and inhibit generation of Aβ peptides.

5-HT4 agonists decrease levels of Aβ peptides and may lower microglial load.

5-HT4 agonists include, e.g., PRX-3140; TD-8954, and TD-5108.

### Activators of Sirtuin Proteins (Sirtuin-Activating Compounds or STAC)

Sirtuins are nicotinamide adenine dinucleotide (NAD+)-dependent protein deacetylases.

Selective Sirtuin 1 (SIRT1) and Sirtuin 2 (SIRT2) activators are of particular interest, preferably SIRT1 activators such as resveratrol, and other polyphenols such as butein, piceatannol, isoliquiritigenin, fisetin, and quercetin.

The most preferred compound is resveratrol.

### HDAC (Histone Deacetylase) Inhibitors

Histone deacetylase inhibitors (HDAC inhibitors, HDIs) are a class of compounds that interfere with the function of histone deacetylase.

Advantageously said compound belongs to the sub-class of non-toxic HDAC2-selective inhibitors selected from the group consisting of trichostatin A, trapoxin B, benzamides, phenylbutyrate, valproic acid, vorinostat, belinostat, LAQ824, panobinostat, entinostat, CI994, and mocetinostat.

### Poly(ADP-Ribose)Polymerase (PARP) Inhibitors

Recently it has been reported that beta-amyloid-induced neuronal death is mediated by poly(ADP-ribose)polymerase (Abeti R et al Brain 2011, 134, 1658-1672).

Advantageously the PARP inhibitor is selected from the group consisting of Olaparib, Rucaparib (also known as HYDAMTIQ), R-503, JPI-289, KCL-440 and from any compound disclosed in WO 2009/054952, WO 2010//056038 and WO 2011/002520, preferably Rucaparib.

Antibodies to Aβ Peptides and β-amyloid

Antibodies to Aβ peptides and β-amyloid decrease levels of Aβ peptides and may reduce microglial load.

Antibodies to Aβ peptides and β-amyloid include, e.g., AAB-001 (bapineuzumab), AAB-002 (a back-up compound to bapineuzumab), AAB-003/PF-05236812 (a humanized 3D6), crenezumab (a humanized monoclonal antibody against human Aβ1-40 and Aβ1-42), ABT-102, ARC029, ARC031, BIIB037 (a fully human immunoglobulin gamma 1 (IgG1) monoclonal antibody against a conformational epitope found on Aβ, AD03/PF-05236812, immune globulin (e.g., Gammagard®), gantenerumab (RG1450), SAR228810 (antibody directed primarily against soluble protofibrillar and fibrillar species of Aβ, which is relatively inactive against Aβ monomers and small oligomeric aggregates), solunezumab.

Immunogenic Peptides that Results in the Production of Antibodies to β-Amyloid

Immunogenic peptides that results in the production of antibodies to β-amyloid and decrease levels of Aβ peptides and may reduce microglial load.

Immunogenic peptides that results in the production of antibodies to β-amyloid include, e.g., vanutide cridificar (ACC-001 (Aβ amino-terminal conjugate)), ACC-002 (amyloid-beta peptide conjugate), AD01 (Aβ amino-terminal mimotope±adjuvant), AD02 vaccine (mimics the N-terminal portion of the Ab 40-42-peptide), CAD105 (Aβ₁₋₅ coupled to Qb virus-like particles); CAD106 (N-terminal Aβ-specific antibodies without an Aβ-specific T-cell response), GSK933776A, V950 (Aβ amino-terminal peptides conjugated to ISCO-MATRIX®.), and UB-311 (an equimolar mixture of 2 synthetic peptides coupled through an oligonucleotide spacer to the N-terminal 14-amino acid fragment of Aβ (Aβ 1-14)).

### Blockers of Oligomers' Aggregation

Blockers of oligomers' aggregation neutralize toxic, low-N Aβ oligomers and prevent them from aggregating. Blockers of oligomers' aggregation therefore decrease levels of Aβ peptides and may reduce microglial load.

Blockers of oligomers' aggregation include, e.g., ELND005 (an inositol stereoisomer that is thought to neutralize toxic, low-N Aβ oligomers and prevent them from aggregating.

### Fibril Formation Inhibitors

Fibril formation inhibitors interfere with the formation of toxic beta-amyloid deposits and fibrils. In certain embodiments, fibril formation inhibitors may also prevent tau protein from forming paired helical filaments.

Fibril formation inhibitors include, e.g., the compound known as Exebryl-1®.

### RAGE Antagonists

RAGE (Receptor for Advanced Glycation End products), first identified a decade ago at COLUMBIA PHYSICIAN & SURGEONS HOSPITAL (P&S), is a molecule that plays a role in numerous diseases, including diabetes, atherosclerosis, and Alzheimer's. RAGE mediates Aβ-induced disturbances in cerebral vessels, neurons, and microglia in AD. RAGE does not instigate the conditions, but escalates the immune and inflammatory response against the body's own cells and tissues and worsens the disease symptoms.

RAGE antagonists may therefore decrease inflammatory response and therefore reduce damage to neurons near Aβ-deposits and fibrils.

RAGE antagonists may also prevent transfer of Aβ, which is generated peripherally, to the brain. Thus, RAGE antagonists decrease levels of Aβ peptides and may reduce microglial load.

RAGE antagonists may bind to the V domain of RAGE and inhibit Aβ40- and Aβ42-induced cellular stress in RAGE-expressing cells.

RAGE antagonists may include, TTP-448; PF-04494700, and FPS-ZM1.

### Pathogenic Tau Level Reducers

Pathogenic tau level reducers compliment and/or facilitate microglial phagocytic activity, and/or prevent or slow down microglial inflammatory activity. Pathogenic tau level reducers include, e.g., tau formation inhibitors, antibodies to truncated tau, peptides that results in antibodies to truncated tau, tau phosphorylation blockers, and tau aggregation inhibitors.

Tau formation inhibitors, include, e.g., R-phenserine.

Antibodies to truncated tau include, e.g., antibodies capable of selectively recognizing a tau truncated at its C-terminus (e.g., at the glutamic acid residue Glu391 or at the aspartic acid residue Asp421) or its N-terminus (e.g., at amino acid Asp13) (e.g., tau1-13, tau14-441, tau14-391, tau391-414, tau1-391, tau1-421, tau14-421, tau14-410, tau391-410, tau14-412, tau391-412, tau 14-383, tau14-381, or tau 14-355, or a fragment of any of the foregoing). These antibodies preferably only recognize, bind or show reactivity with truncated tau, but do not recognize, bind or show reactivity with a normal tau protein (e.g., a full length untruncated htau40). The antibody may, e.g., be selected from the group consisting of MN423, TauC3, Tau12, 5A6, DC11, anti-cleaved-Tau (ASP421), clone C3, structurally or functionally similar antibodies. In certain embodiments, the antibody is TauC3, or a structurally and/or functionally similar antibody.

Peptides that results in antibodies to truncated tau include, e.g., tau1-13, tau14-441, tau14-391, tau391-414, taut-391, tau1-421, tau14-421, tau114-410, tau391-410, tau14-412, tau391-412, tau14-383, tau14-381, tau143-355, or an immunogenic fragment of any of the foregoing. In certain embodiments, peptide that results in the production of antibodies to truncated tau include an epitope of an antibody capable of selectively recognizing a tau truncated at its C-terminus (e.g., at the glutamic acid residue Glu391 or at the aspartic acid residue Asp421) or its N-terminus (e.g., at amino acid Asp13) (e.g., tau1-13, tau14-441, tau14-391, tau391-414, tau1-391, tau1-421, tau14-421, tau14-410, tau391-410, tau14-412, tau391-412, tau 14-383, tau14-381, or tau 14-355, or a fragment of any of the foregoing). For example, the peptide may include an epitope of MN423, TauC3, Tau12, 5A6, DC11, anti-cleaved-Tau (ASP421), clone C3, structurally or functionally similar antibodies.

Phosphorylation blockers more commonly referred to as kinase inhibitors, lower the amount of unbound tau that is available for aggregation and possibly slow the rate of aggregation. Phosphorylation blockers include, e.g., davunetide, synthase kinase (GSK)-3 beta and cyclin-dependent kinase-5.

Tau aggregation inhibitors inhibit aggregation of tau. Tau aggregation inhibitors include, e.g., methylthioninium chloride (e.g., Trx-0237 (LMTX™)) and antibodies selective for pathogenic tau dimers and oligomers. Antibodies selective for pathogenic tau dimers and oligomers, include, e.g., TOC-1 antibody.

Tau level reducers may facilitate removal of A13, reduce microglial load and potentiate the actions of 1-phenylalkanecaroxylic acids (e.g., CHF 5074) and/or Aβ peptides level reducers.

Tau level reducers may also decrease side effects associated with, e.g., Aβ peptides level reducers.

Microtubule Stabilizers

Tau is a microtubule (MT)-stabilizing protein that is altered in Alzheimer's disease (AD) and other tauopathies. Tau-mediated loss of MT stability may contribute to disease progression

Microtubule stabilizers may compliment microglial phagocytic activity, and/or slow down microglial inflammatory activity.

Microtubule stabilizers include, e.g., DBMS-241027 (Epothilone D).

### Agents Capable of Removing Atherosclerotic Plaques

Agents capable of removing atherosclerotic plaques may reduce microglial load and compliment and/or facilitate microglial phagocytic activity, and/or slow down microglial inflammatory activity.

Agents capable of removing atherosclerotic plaques include, e.g., BET protein inhibitors (e.g., RVX-208).

RVX-208 functions by removing atherosclerotic plaque via reverse cholesterol transport (RCT), the natural process through which atherosclerotic plaque is transported out of the arteries and removed from the body by the liver. RVX-208 also increases production of Apolipoprotein A-I (ApoA-I), a building block of functional high-density lipoprotein (HDL) particles and the type required for RCT. These newly produced, functional HDL particles are flat and empty and can efficiently remove plaque and stabilize or reverse atherosclerotic disease.

Agents capable of removing atherosclerotic plaques may therefore compliment and facilitate actions of 1-phenylalkanecaroxylic acids (e.g., CHF 5074), Aβ peptides level reducers, pathogenic tau level reducers, and microtubule stabilizers.

Agents that Lower Circulating Levels of β-Amyloid and Tau

Agents that lower circulating levels of Aβ peptides and tau may reduce microglial load and compliment and/or facilitate microglial phagocytic activity, and/or slow down microglial inflammatory activity. These agents include, e.g., nomethiazoles (e.g., Sgc-1061).

Agents that lower circulating levels of Aβ peptides and tau may therefore compliment and facilitate actions of 1-phenylalkanecaroxylic acids (e.g., CHF 5074), Aβ peptides level reducers, pathogenic tau level reducers, and microtubule stabilizers, and agents capable of removing atherosclerotic plaques.

### Modulators of Autophagy

Modulators of autophagy increase autophagy, a process that clears away unwanted protein aggregates. Modulators of autophagy compliment and/or facilitate microglial phagocytic activity. Modulators of autophagy include, e.g., LNK-754, peroxisome proliferator-activated receptor, alpha/gamma agonists, and agents that reduce glucocorticoid activity.

Alpha/gamma agonists enhanced the microglial uptake/phagocytosis of Aβ in a PPARγ-dependent manner, which subsequently results in a reduction of cortical and hippocampal Aβ levels. Alpha/gamma agonists may improve spatial memory performance. An exemplary alpha/gamma agonist is DSP-8658.

Agents that Reduce Glucocorticoid Activity

Evidence suggests that excessive glucocorticoid activity may contribute to AD and age-associated memory impairment. It may also inhibit microglial phagocitotic activity.

Agents that reduce glucocorticoid activity should therefore compliment or facilitate microglial phagocitotic activity and include, e.g., selective inhibitor of 11-beta-hydroxysteroid dehydrogenase type 1 (11β-hydroxysteroid dehydrogenase type-1 (HSD1), which regulates conversion of glucocorticoids from inactive to active forms.

An exemplary agent that reduces glucocorticoid activity is ABT-384.

### Neurotransmitter Level Regulators

Imbalance of neurotransmitters may lead to microglial dysfunction, decrease microglial phagocytic activity and increase microglial inflammatory activity.

Neurotransmitter level regulators modulate or increase levels of neurotransmitters (e.g., acetylcholine, dopamine, histamine, serotonin, norepinephrine), may lower inflammation, may increase microglial recruitment and phagocytic effects, may prevent or slow down microglia inflammatory activity, and/or may help maintain/restore cognitive function and functional deficits of Alzheimer's disease, and/or slow down decline in cognitive functions and functional deficits in AD.

Neurotransmitter level regulators include, e.g., acetylcholinesterase inhibitors, butyrylcholinesterase inhibitors, MOA B inhibitors, serotonin receptor antagonists, histamine receptor 3 (H3) antagonists, and NMDA receptor antagonists.

### Acetylcholinesterase Inhibitors

Acetylcholinesterase inhibitors increase levels of acetylcholine.

Acetylcholinesterase inhibitors include, e.g., methanesulfonyl fluoride (SeneXta Therapeutics), ladostigil (Avraham), rilapladib (GlaxoSmithKline), phenserine (QR Pharma); huperzine A (Xel pharmaceuticals).

### Butyrylcholinesterase Inhibitors

Butyrylcholinesterase inhibitors increase levels of acetylcholine. An exemplary butyrylcholinesterase inhibitor is bisnorcymserine (BNC).

### MOA B Inhibitors

MOA B enzyme breaks down dopamine in the brain and contributes to the production of free radicals. Brains of AD patients exhibit up-regulation of MAO-B expression.

Selective MAO-B inhibitors may therefore treat or slow down progression of AD.

Selective MAO-B inhibitors include, e.g., RG1577; EVT 302, and selegiline.

### Serotonin Receptor Antagonists

Serotonin levels correlate to clinical manifestations of AD and appear to be involved in dysfunctions of multiple neurotransmitter pathways.

Serotonin receptor 6 (5-HT6) is a subtype localized almost exclusively in the CNS. The 5-HT6-receptor is expressed in brain regions involved in cognition, such as the cortex and the hippocampus, and modulates activity of multiple neurotransmitter system.

Blockade of 5-HT6 receptors leads to enhancements of cholinergic, glutamatergic, noradrenergic, and dopaminergic neurotransmission, together with learning-associated neuronal remodeling, and an improvement of cognitive performance in a wide variety of learning and memory paradigms.

Serotonin receptor antagonist include e.g., SB-742457, AVN 101, AVN322, AVN 397, SB-742457, GSK742457, LU AE58054, PF-05212377, and SYN-120.

### Histamine Receptor 3 (H3) Antagonists

H3 antagonists enhance in-vivo release of neurotransmitters (e.g., acetylcholine, dopamine, and histamine).

H3 antagonists include, e.g., ABT-288, AZD5213, GSK239512, irdabisant (CEP-26401), and SAR1180894.

### NMDA Receptor Antagonists

NMDA receptor antagonists help block the activity of the neurotransmitter glutamate by binding to N-methyl-D-aspartate (NMDA) receptors on the surface of brain cells. Glutamate, at appropriate levels, plays an important role in learning and memory. If glutamate levels are too low, cognitive problems may develop. If levels are too high, glutamate overstimulates nerve cells and may lead to cell death.

NMDA antagonists include, e.g., memantine (Namenda), and ASP0777.

Neurotransmitter level regulators therefore increase levels of neurotransmitters (e.g., acetylcholine, dopamine, histamine, serotonin, norepinephrine), may lower inflammation, may increase microglial recruitment and phagocytic effects, may prevent or slow down microglia inflammatory activity, and/or may help maintain/restore cognitive function and functional deficits of Alzheimer's disease, and/or slow down decline in cognitive functions and functional deficits in AD.

### GABA(A) α5 Receptors Inhibitors

GABA(A) α5 receptors mediate tonic inhibition of principal neurons. Condition of excess activity in the hippocampal formation is observed in the aging brain and in conditions that confer additional risk during aging for AD. Antagonism of GABA(A) α5 receptors should therefore slow down the progression of AD and may potentiate actions of 1-phenylalkanecaroxylic acids (e.g., CHF 5074), Aβ peptides level reducers, pathogenic tau level reducers, microtubule stabilizers, agents capable of removing atherosclerotic plaques, agents that lower circulating levels of Aβ and tau, autophagy modulators, and neurotransmitter regulators.

GABA(A) α5 receptors antagonists, include, e.g., RG1662, 6,6-dimethyl-3-(3-hydroxypropyl)thio-1-(thiazol-2-yl)-6,7-dihydro-2-benzothiophen-4(5H)-one] and methyl 3,5-diphenylpyridazine-4-carboxylate.

Additional Agents that Help Maintain and/or Restore Cognitive Function and Functional Deficits of AD, and/or Slow Down Decline in Cognitive Functions and Functional Deficits in AD

Additional agents that may help maintain/restore cognitive function and functional deficits of Alzheimer's disease, and/or slow down decline in cognitive functions and functional deficits in AD include, e.g., alpha-4 beta-2 nicotinic receptor modulators, M1 selective muscarinic agonists, alpha4/beta2 neuronal nicotinic receptor agonists, α-7 nicotinic acetylcholine receptor (α7-nAChR) allosteric modulators, insulin sensitizers, calpain inhibitors, neurotrophic agents, and nicotinic receptor agonists.

### Alpha-4 Beta-2 Nicotinic Receptor Modulators

Alpha-4 beta-2 nicotinic receptor modulators reduce inflammatory neurotoxicity. Alpha-4 beta-2 nicotinic receptor modulators may therefore facilitate and compliment actions and/or reduce side effects of 1-phenylalkanecaroxylic acids (e.g., CHF 5074), AP peptides level reducers, pathogenic tau level reducers, microtubule stabilizers, agents capable of removing atherosclerotic plaques, agents that lower circulating levels of Aβ and tau, autophagy modulators, neurotransmitter regulators, and/or GABA(A) α5 receptors inhibitors.

Alpha-4 beta-2 nicotinic receptor modulators include, e.g., ABT-560.

### M1 Selective Muscarinic Agonists

Aβ peptides may impair the coupling of M1 muscarinic ACh receptors (mAChRs) with G proteins. This impairment may lead to decreased signal transduction, to a reduction in levels of trophic amyloid precursor proteins (APPs), and to generation of more beta-amyloids that can also suppress ACh synthesis and release, aggravating further the cholinergic deficiency.

M1 selective muscarinic agonists may therefore promote the nonamyloidogenic APP processing pathways and decrease tau protein phosphorylation, facilitate and compliment actions and/or reduce side effects of 1-phenylalkanecaroxylic acids (e.g., CHF 5074), Aβ peptides level reducers, pathogenic tau level reducers, microtubule stabilizers, agents capable of removing atherosclerotic plaques, agents that lower circulating levels of Aβ and tau, autophagy modulators, neurotransmitter regulators, and/or GABA(A) α5 receptors inhibitors.

M1 selective muscarinic agonists may, e.g., be MCD-386, AF102B, or AF150(S).

### Alpha4/Beta2 Neuronal Nicotinic Receptor Agonists

Nicotinic acetylcholine receptors (nAChRs) are ligand-gated ion channels that are widely distributed in the human brain where they have a modulatory function associated with numerous transmitter systems. Reductions in nAChR density have been identified in a number of neurodegenerative disorders including Alzheimer's disease (AD), dementia with Lewy bodies (DLB), and Parkinson's disease (PD) The major nAChR subtypes present in the mammalian brain are 7 and 4 2.

Stimulation of alpha4beta2 nicotinic acetylcholine receptors inhibits beta-amyloid toxicity. Alpha4/beta2 neuronal nicotinic receptor agonists may therefore facilitate and compliment actions and/or reduce side effects of 1-phenylalkanecaroxylic acids (e.g., CHF 5074), Aβ peptides level reducers, pathogenic tau level reducers, microtubule stabilizers, agents capable of removing atherosclerotic plaques, agents that lower circulating levels of Aβ and tau, autophagy modulators, neurotransmitter regulators, and/or GABA(A) α5 receptors antagonists.

Alpha4/beta2 neuronal nicotinic receptor agonists may, e.g., be AZD1446, AZD3480 (isproniclidine), 3-Bromocytisine, acetylcholine, cytosine, epibatidine, nicotine, A-84,543, A-366,833, ABT-418, altinicline, dianicline, ispronicline, pozanicline, rivanicline, tebanicline, TC-1827, varenicline, sazetidine A, or N-(3-pyridinyl)-bridged cyclic diamines.

α-7 Nicotinic Acetylcholine Receptor (α7-nAChR) Allosteric Modulators

α7-Nicotinic acetylcholine receptors (α7 nAChRs) play a role in cognitive function. Positive allosteric modulators (PAMs) amplify effects of α7 nAChR agonist and could provide an approach for slowing progression of cognitive symptoms of AD, facilitate and compliment actions and/or reduce side effects of 1-phenylalkanecaroxylic acids (e.g. CHF 5074), Aβ peptides level reducers, pathogenic tau level reducers, microtubule stabilizers, agents capable of removing atherosclerotic plaques, agents that lower circulating levels of Aβ and tau, autophagy modulators, neurotransmitter regulators, and/or GABA(A) α5 receptors antagonists.

An exemplary α7-Nicotinic acetylcholine receptors may, e.g., be ABT-126.

### Insulin Sensitizers

Insulin sensitizers may improve cognitive function and in some circumstances help slow the rate of cognitive decline in AD, facilitate and compliment actions and/or reduce side effects of 1-phenylalkanecaroxylic acids (e.g., CHF 5074), Aβ peptides level reducers, pathogenic tau level reducers, microtubule stabilizers, agents capable of removing atherosclerotic plaques, agents that lower circulating levels of Aβ and tau, autophagy modulators, neurotransmitter regulators, and/or GABA(A) α5 receptors inhibitors.

Insulin sensitizers include, e.g., Metformin, MSDC-0160, rosiglitazone, and pioglitazone.

### Calpain Inhibitors

Calpain is a protein belonging to the family of calcium-dependent, non-lysosomal cysteine proteases (proteolytic enzymes) expressed ubiquitously in mammals and many other organisms. Calpain inhibitors may therefore regulate neurological functions, facilitate and compliment actions and/or reduce side effects of 1-phenylalkanecaroxylic acids (e.g., CHF 5074), Aβ peptides level reducers, pathogenic tau level reducers, microtubule stabilizers, agents capable of removing atherosclerotic plaques, agents that lower circulating levels of Aβ and tau, autophagy modulators, neurotransmitter regulators, and/or GABA(A) α5 receptors antagonists.

A calpain inhibitor may be a compound disclosed in WO 2012/076639 or the compound known as ABT-957.

### Neurotrophic Agents

Neurotrophic agents include, e.g., CERE-110 (Nerve growth factor-beta stimulator), and T-817MA [1-{3-[2-(1-Benzothiophen-5-yl)ethoxy]propyl}-3-azetidinol maleate].

### Nicotinic Receptor Agonists

It is suggested that both pre- and postsynaptic α7 nAChRs modulate neurotransmitter release in the brain through Ca2+-dependent mechanisms, and that the α7 nAChRs play a role in regulating neuronal growth and differentiation in the developing CNS. Nicotinic receptor agonists may therefore facilitate and compliment actions and/or reduce side effects of 1-phenylalkanecaroxylic acids (e.g., CHF 5074), Aβ peptides level reducers, pathogenic tau level reducers, microtubule stabilizers, agents capable of removing atherosclerotic plaques, agents that lower circulating levels of Aβ and tau, autophagy modulators, neurotransmitter regulators, and/or GABA(A) α5 receptors inhibitors.

Nicotinic receptor agonists include, e.g., AZD1446, BMS-933043, EVP-6124, and TC-5619.

Thus, the neuroprotective agents may, e.g., be selected from the group consisting of antibodies to Aβ, neurotoxic tau, or any one or more neuroprotective agents known to those having ordinary skill in the art. Examples of such neuroprotective agents include, but are not limited to the following: AAB-002 (amyloid beta-protein inhibitor mAβ) from Janssen Alzheimer Immunotherapy/Pfizer, AAB-003/PF-05236812 (amyloid beta-protein inhibitor mAβ) from Janssen Alzheimer Immunotherapy/Pfizer, ABT-126 (alpha-7 neuronal nicotinic receptor antagonist) from Abbott Laboratories, ABT-288 (neurotransmitter receptor modulator) from Abbott Laboratories, ABT-384 from Abbott Laboratories, ABT-560 (alpha-4 beta-2 nicotinic receptor modulators) from Abbott Laboratories, ABT-560 (alpha-4 beta-2 nicotinic receptor modulators) from Abbott Laboratories, ACC-002 (amyloid-beta peptide conjugate) from Janssen Alzheimer Immunotherapy/Pfizer, AD02 vaccine from Affiris/GlaxoSmithKline, AD03 vaccine from Affiris/GlaxoSmithKline, ADS-8704 (donepezil/memantine) from Adamas Pharmaceuticals, APH-0703 from Aphios, ARC029 (soluble amyloid reducing/clearing agent) from Archer Pharmaceuticals, ARC031 (soluble amyloid reducing/clearing agent) from Archer Pharmaceuticals, ASP0777 from Astellas Pharma US, AVN 101 (serotonin 6 receptor antagonist) from Avineuro Pharmaceuticals, AVN 322 (serotonin 6 receptor antagonist) from Avineuro Pharmaceuticals, AVN 397 from Avineuro Pharmaceuticals, AZD1446 (alpha4/beta2 neuronal nicotinic receptor agonist) from AstraZeneca/Targacept, AZD3480 (ispronicline) from AstraZeneca/Targacept, AZD4694 (fluorine-18 labeled precision radiopharmaceutical) from Navidea Biopharmaceuticals, AZD5213 (histamine-3 receptor antagonist) from AstraZeneca, β secretase inhibitor from Eli Lilly, BAN2401 (amyloid beta-protein inhibitor) from BioArtic Neuroscience/Eisai, bapineuzumab subcutaneous (AAB-001) from Janssen Alzheimer Immunotherapy/Pfizer, BCI-632 from BrainCells, BCI-838 from BrainCells, BIIB037 (amyloid beta-protein inhibitor) from Biogen Idec, bisnorcymserine (BNC) from QR Pharma, BMS-241027 (microtubule stabilizer) from Bristol-Myers Squibb, BMS-708163 (avagacestat) from Bristol-Myers Squibb, BMS-932481 (gamma secretase modulator) from Bristol-Myers Squibb, BMS-932481 (gamma secretase modulator) from Bristol-Myers Squibb, CAD106 (amyloid beta-protein inhibitor) from Novartis Pharmaceuticals, CERE-110 (AAV-NGF gene therapy) from Ceregene, crenezumab (anti-Abeta) from Genentech, CTS-21166 (β-secretase inhibitor) from Astellas Pharma US/CoMentis, CX717 from Cortex Pharmaceuticals, davunetide intranasal from Allon Therapeutics, docosahexaenoic acid (DHA) Martek Biosciences, DSP-8658 (PPAR α/γ agonist) from Sunovion Pharmaceuticals, E2212 (amyloid precursor protein secretase modulator) from Eisai, E2609 (BACE1 protein inhibitor) from Eisai, ELND005 (amyloid beta-protein inhibitor) Elan/Transition Therapeutics, EVP-0962 (amyloid precursor protein secretase modulator) from EnVivo Pharmaceuticals, EVP-6124 (α7-nAChR agonist) from EnVivo Pharmaceuticals, Exebryl-1® from ProteoTech, F18-florbetaben (molecular imaging agent) from Piramal Healthcare, F18-flutemetamol (PET imaging agent) from GE Healthcare, Gammagard® immune globulin intravenous (human), 10% solution from Baxter Healthcare, gantenerumab (RG1450) from Roche, GSK239512 from GlaxoSmithKline, GSK742457 (5HT6 antagonist) from GlaxoSmithKline, GSK933776A (anti-B amyloid mAb) from GlaxoSmithKline, HPP-854 (BACE1 inhibitor) from High Point Pharmaceuticals, human immunoglobulin (intravenous) from Grifols USA, immune globulin high dose from Octapharma USA, irdabisant (CEP-26401) from Cephalon, LMTX (TRx-0237) from TauRx Pharmaceuticals, LNK-754 from Link Medicine, LU AE58054 from Lundbeck, MCD-386/glycopyrrolate from Mithridion, MK-3134 from Merck, MK-3328 (PET tracer) from Merck, MK-8931 (BACE1 inhibitor) from Merck, MSDC-0160 from Metabolic Solutions Development Company, NIC5-15 from Humanetics, PF-05212377 (SAM-760) from Pfizer, the antioxidant compound indole-3-propionic acid (Oxigon™), pioglitazone from Takeda Pharmaceuticals U.S.A./Zinfadel Pharmaceuticals, Posiphen™ R-phenserine from QR Pharma, PRX-3140 (5-HT4 partial agonist) from Nanotherapeutics, RG1577 (MAO-B inhibitor) from Roche, RG1662 (GABAA α5 receptor modulator) from Roche, rilapladib from GlaxoSmithKline/Human Genome Sciences, RVX-208 (BET protein inhibitor) from Resverlogix, SAR110894 (H3 antagonist) from Sanofi US, SAR228810 from Sanofi US, sGC-1061 from sGC Pharma, solanezumab from Eli Lilly, ST-101 from Sonexa Therapeutics, SYN-120 from Biotie Therapies, T-817MA from Toyama Chemical, TC-5619 from Targacept, TD-8954 (5-HT4 agonist) from Theravance, TTP-448 (RAGE antagonist) from TransTech Pharma, UB-311 (amyloid beta protein inhibitor vaccine) from United Biomedical, V950 vaccine from Merck, vanutide cridificar (ACC-001).

In certain embodiments, the neuroprotective agent is velusetrag (TD-5108) from Theravance, VI-1121 from VIVUS, XEL 001HP (transdermal patch) from Xel Pharmaceuticals, and combinations of any or all of the foregoing.

In certain embodiments, the neuroprotective agent may comprise antibodies to Aβ, neurotoxic tau, or any one or more neuroprotective agents known to those having ordinary skill in the art.

### Alzheimer's Disease

AD is a common chronic progressive neurodegenerative disease in which there is neuronal cell degeneration and an irreversible loss of cognitive and behavioral functions.

AD can last for over 10 years, advancing from mild symptoms to extremely severe manifestations. AD is said to afflict approximately 10% of the population over the age of 65, and more than 30% of the population over the age of 80.

The predominant initial clinical symptom of AD is the impairment of memory, although a wide range of other higher functions, such as personality and judgment, are also affected. Yet in very early, asymptomatic AD, pre-tangle tau aggregates may be or are already present in the entorhinal cortex and hippocampal regions of the brain. These are the same regions where neuronal degeneration and loss of neuronal cells occur later as the disease progresses. With time, Tau tangles also form in the parieto-temporal and frontal region of the cortex, resulting in neuronal dysfunction and correlating with the worsening of clinical symptoms.

The severity and progression of AD is generally characterized by Braak stages, using a scheme described by Braak and Braak in Tau Aggregates Correlate with Cognitive Impairment During the 1990s. Braak graded the presence, distribution and density of Tau tangles in the brain and defined six distinct stages of AD progression (“Braak stages”). Braak stage is a measure of where and how many tangles there are in the brain.

Braak stage I is the point at which tau protein starts to clump into tau tangles. At this stage, the tau tangles have begun to form in the transitional entorhinal region of the brain, which is a “relay station” between the cortex and the hippocampus, and is critical for memory. There are no external symptoms at this stage, and it may take a number of years (e.g., 10 to 15 years) after this stage before any symptoms (e.g., dementia) are noticed.

By Braak stage II, tau tangles have accumulated further and have caused some neurons to burst apart and die. At this stage, the tau tangles are much more extensive in the transitional entorhinal region and have begun to kill neurons there. At the same time, tau protein began to accumulate in the brain's hippocampus and neocortex, but has not yet formed tangles there. However, mental testing at this stage still shows minimal impairment.

By Braak stage III, the tau tangles have begun to cause extensive neuronal death. A proposed mechanism for neuronal death is that the tau tangles grow out of control. Tau tangles fill up the neuron, causing its membrane to burst. Although, at this stage, tau tangles and neuronal death have likely caused some memory impairment, only about ten percent of patients at this stage would be diagnosed as suffering from dementia.

By Braak stage IV, even though the tau tangles still occupy only a small portion of the brain, tau tangles have caused significant memory and cognitive impairment. By this stage, the tau tangles have formed extensively in the transitional entorhinal region and the hippocampus, where they have caused neuronal death, and the tangles are starting to form in neo-cortex. Neo-cortex is the largest part of the brain and is involved in higher functions such as sensory perception, conscious thought and language. Seventy percent of patients with this level of tangles in their brain would be diagnosed as suffering from dementia.

By Braak stage V, the tau tangles have caused extensive neuronal death, giving rise to severe memory and cognitive impairment. Tangles have formed extensively in the transitional entorhinal region, the hippocampus (which is critical for memory), and the neo-cortex. About eighty percent of patients with this level of tangles would be diagnosed as suffering from moderate to severe dementia. They would be completely unable to take care of themselves and will have trouble recognizing family members.

AD is also characterized by the extracellular accumulation of plaques composed of amyloid β (Aβ), the intracellular accumulation of the microtubule-associated protein Tau into neurofibrillary tangles (NFTs), and extracellular tau (dystrophic neuritis). Aβ plaques are generally believed to be preceded by formation of extracellular soluble pathogenic Aβ forms, including dimers, trimers, and oligomers, fibrils. There also appears to be a potential link between amyloid beta aggregation and Tau pathology.

NFTs are composed of Tau aggregates in the form of paired helical filaments and straight filaments. Unlike Aβ plaques, the spatial and temporal progression of NFTs positively correlates with the progression of clinical symptoms.

Although the spatiotemporal distribution of NFTs correlates with neuron loss and cognitive impairment in AD, current evidence suggests that NFTs may not be the primary form of Tau underlying neuronal dysfunction. Consequently, it has been proposed that prefibrillar Tau aggregates may be responsible for a large part of disease-related neurotoxicity.

In AD, Tau is cross-linked by transglutaminases and products of lipid peroxidation such as hydroxynonenal (product of AA peroxidation), and these modifications may even promote Tau aggregation by stabilizing AD-associated Tau conformations such as Alz-50 (See, Sayre, L. M., Zelasko, D. A., Harris, P. L., Perry, G., Salomon, R. G., and Smith, M. A. (1997) J. Neurochem. 68, 2092-2097; Liu, Q., Smith, M. A., Avila′, J., DeBernardis, J., Kansal, M., Takeda, A., Zhu, X., Nunomura, A., Honda, K., Moreira, P. I., Oliveira, C. R., Santos, M. S., Shimohama, S., Aliev, G., de la Torre, J., Ghanbari, H. A., Siedlak, S. L., Harris, P. L., Sayre, L. M., and Perry, G. (2005) Free Radic. Biol. Med. 38, 746-754; Appelt, D. M., and Balin, B. J. (1997) Brain Res. 745, 21-31; Balin, B. J., and Appelt, D. M. (2000) Methods Mol. Med. 32, 395-404; Dudek, S. M., and Johnson, G. V. (1993) J. Neurochem. 61, 1159-1162; and Singer, S. M., Zainelli, G. M., Norlund, M. A., Lee, J. M., and Muma, N. A. (2002) Neurochem. Int.40, 17-30, all of which are incorporated herein by reference in their entireties). Although it is likely that Tau dimerization occurs under physiological conditions, the process may become dysregulated in disease. Formation of stable cross-links may be one mechanism by which the equilibrium shifts away from soluble, monomeric Tau toward Tau aggregates.

Additionally, Tau appears to be necessary for (contribute to) AP-induced neurotoxicity in cell culture and transgenic mouse models β-5). Tau inclusions are also found in other tauopathies that lack Aβ pathology, including Pick's disease, corticobasal degeneration, and progressive supranuclear palsy. Notably, mutations in the tau gene cause some forms of frontotemporal dementia, signifying that Tau dysfunction is sufficient to cause neurodegeneration.

### Administration of 1-Phenylalkanecarboxylic Acids and Neuroprotective Agents

1-Phenylalkanecarboxylic acids and neuroprotective agents used in the methods of present invention may be administered orally, intranasally, by a subcutaneous injection, intramuscular injection, IV infusion, transcutaneously, buccally, and may be included into pharmaceutical compositions for intranasal, subcutaneous, intramuscular injection, IV, transcutaneously, buccal or oral administration, as described in more detail below.

### Antibodies

Isolated antibodies which may be used the present invention (e.g., non-naturally occurring antibodies or genetically engineered antibodies) include glycoproteins made up of light (L) and heavy (H) polypeptide chains, or segments of any of the foregoing. L and H chains are subdivided into variable and constant regions. The variable regions are responsible for antigen-binding.

In certain preferred embodiments, the antibody is TOC-1, or an antibody having the variable region of the heavy chain which is homologous to the variable region of the TOC-1 antibody.

In certain embodiments, isolated antibodies of the invention are capable of and selectively recognize prefibrillar pathological or neurotoxic tau and precursors comprising at least two tau proteins, or fragments thereof, cross-linked to each other, directly or through a linker (e.g., B4M), at one or more cysteine residues.

In certain other preferred embodiments, the isolated antibodies selectively recognize a pathogenic dimer comprising two tau monomers cross-linked to each other, directly or through a linker. The dimer is formed in-vitro and has a conformation which may be representative of a pathogenic conformation of a dimer formed in-vivo which may be responsible for initiating a cascade of events in which normal tau becomes directly neurotoxic or/and a chain of aggregation events leading to pathogenic prefibrillar tau oligomers, and eventually formation of NFTs. In the preferred embodiments, at least one of the cross-links between the individual tau monomers of the dimer formed in-vitro is not a disulfide bridge between cysteines of the tau monomers.

The linker may be an agent which has a sulfhydryl (SH) group and is capable of reacting with available cites upon UV illumination. The linker may, e.g., be selected from the group consisting of B4M, PEAS (N-((2-pyridyldithio)ethyl)-4-azidosalicylamide), succinimidyl trans-4-(maleimidylmethyl)cyclohexane-1-carboxylate (SMCC), 3-(2-pyridyldithio)propionate (SPDP), 2,5-Pyrrolidinedione, 1-[1-oxo-3-(2-pyridinyldithio)propoxy], succinimidyl acetylthioacetate (SATA), N-((2-pyridyldithio)ethyl)-4-azidosalicylamide), or the like. In the preferred embodiments, the linker is B4M.

The antibodies of the invention may specifically recognize a pathogenic conformation of the prefibrillar pathological or neurotoxic tau and precursors. In the preferred embodiments, this conformation is the conformation induced by cross-linking tau monomers as described in the present specification.

In certain preferred embodiments, the antibodies of the invention are selective for the epitope comprising a fragment comprising or consisting of amino acid residues 221-228, or a portion thereof, of hTau40.

In certain preferred embodiments of the invention, the antibodies of the invention (i) inhibit, reduce, clear and/or eliminate formation of prefibrillar pathological tau aggregates, (ii) inhibit, reduce, clear and/or eliminate prefibrillar pathological aggregation of Tau, and/or (iii) prevent the formation of neurofibrillary tangles and/or increase clearance of the neurofibrillary tangles, all without affecting the biological functions of normal tau proteins. These antibodies do not affect the biological functions of normal tau proteins because these antibodies are selective for prefibrillar pathological or neurotoxic tau and precursors (i.e., they do not bind or do not sufficiently bind normal tau proteins to affect their biological function, e.g., when tested at saturating levels of antibody-immunogen binding).

The present invention is additionally directed to combination therapy via the administration of pharmaceutical compositions comprising 1-phenylalkanecarboxylic acids together with a pharmaceutical composition(s) which include a (e.g., recombinant) antibody that discriminates between an Aβ peptide and the β-amyloid protein precursor from which it is proteolytically derived, and is also referred to as an “antisenilin”. By “antisenilin” is meant a molecule which binds specifically to a terminus/end of an Aβ peptide to slow down or prevent the accumulation of amyloid-β peptides in the extracellular space, interstitial fluid and cerebrospinal fluid and the aggregation into senile amyloid deposits or plaques and to block the interaction of Aβ peptides with other molecules that contribute to the neurotoxicity of Aβ. By providing antisenilins in the extracellular space, interstitial fluid and cerebrospinal fluid, where soluble Aβ peptides are present, the formation of soluble antisenilin-Aβ complexes are promoted which are cleared from the central nervous system by drainage of the extracellular space, interstitial fluid and cerebrospinal fluid into the general blood circulation through the arachnoid villi of the superior sagittal sinus. In this manner, soluble Aβ peptides are prevented from accumulating in the extracellular space, interstitial fluid and cerebrospinal fluid to form amyloid deposits and/or to induce neurotoxicity. Furthermore, clearance of soluble amyloid-β peptides in accordance with the present invention is expected to reduce the inflammatory process observed in proteinopathies such as Alzheimer's Disease by inhibiting, for example, amyloid-β-induced complement activation and cytokine release, and block also the interaction of Aβ with cell surface receptors such as the RAGE receptor. Neuritic plaques are mainly composed of aggregates of a peptide with 39-43 amino acid residues known as β-amyloid (βA), and, depending on the numbers of amino acids, Aβ39, Aβ40, Aβ42 and Aβ43.

In certain preferred embodiments, the antibody is a (e.g., recombinant) antibody molecule end-specific for the N-terminus or the C-terminus of an amyloid-β peptide, e.g., Aβ₁₋₄₂

In certain other preferred embodiments, the antibody is a (e.g., recombinant) antibody is specific for a truncated tau proteins selected from the group consisting of hTau40 truncated at its C-terminus at the glutamic acid residue Glu391, hTau40 truncated at the aspartic acid residue Asp421, hTau40 truncated at its N-terminus at the aspartic acid residue Asp13, proteins homologous to hTau40 truncated at its C-terminus at the glutamic acid residue Glu391, proteins homologous to hTau40 truncated at the aspartic acid residue Asp421, and proteins homologous to hTau40 truncated at its N-terminus at the aspartic acid residue Asp13, but shows no binding and/or reactivity to full length hTau40.

The antibodies of the invention include polyclonal and monoclonal antibodies.

The antibodies of the invention also include recombinant antibodies.

The antibodies of the invention further include, e.g., chimeric antibodies, humanized antibodies, human antibodies, murine antibodies, camelid antibodies, fragments of any of the foregoing (e.g., Fc fragments, Fab fragments, subfragments of any of the foregoing, etc.), and hybrid antibodies (e.g., biselective or bifunctional antibodies).

The antibodies of the invention specifically include single chain antibodies (e.g., camelid antibodies). Single chain antibodies have a potential to penetrate the brain more readily than full-sized immunoglobulins and are less likely to induce unwanted immune reactions.

Any of the antibodies mentioned above may be an IgM or an IgG antibody, or a fragment of any of the foregoing. IgM and IgG antibodies are made up of four polypeptide chains linked together by disulfide bonds. The four chains of whole (intact) IgM and IgG antibodies are two identical heavy chains referred to as H-chains and two identical light chains referred to as L-chains.

In the embodiments where the antibody is an IgG antibody, the IgG antibody may be obtained by an immunoglobulin class switching by rearrangement of a gene of an IgM antibody according to the present invention which will result in the elaboration of IgG antibodies of the same antigenic specificity as the IgM antibody.

In yet another embodiment of the invention, the antibodies of the present invention may be conjugated to a cytoprotective agent directly or through a linker. The cytoprotective agent may be an antioxidant (e.g., melatonin or a different agent capable of cross-linking. The cytoprotective agent should be recognized as safe (GRAS) by the United States Food and Drug Administration (“FDA”). The linker may be selected from the group comprising or consisting of a hydrazine linker, a disulfite linker, a thioether linker, a peptide linker, or the like. In certain embodiments, the antibody is selective for ATau, and the cytoprotective agent is melatonin.

In an additional embodiment of the invention, the antibodies of the present invention may be conjugated to an agent which may improve antibody's ability to cross the BBB and is generally recognized as safe (GRAS) by the United States Food and Drug Administration (“FDA”). The agent which facilitates or improves antibody's ability to cross the BBB may be conjugated to the antibody directly or through a linker comprising or consisting of a hydrazine linker, a disulfite linker, a thioether linker, a peptide linker, or the like. The agent which facilitates or improves antibody's ability to cross the BBB may comprise or consists of transferrin, insulin receptor bispecific antibodies or other targeting signals.

Antibodies of the invention are suitable for crossing BBB and for administration, e.g., by a subcutaneous injection, nasal administration, intramuscular injection, IV infusion, transcutaneous injection, buccal administration, oral administration, or as described in more detail below.

### Pharmaceutical Compositions

Pharmaceutical formulations in accordance with the present invention may comprise (i) an active agent comprising a therapeutically effective amount of a 1-phenylalkanecarboxylic acid and/or one or more additional neuroprotective agents as described herein.

The pharmaceutical composition of the present invention is in certain embodiments directed to a single active agent, CHF 5074 in an amount from about 50 mg to about 550 mg, and preferably from about 200 mg to about 400 mg. The amount of CHF 5074 contained in the dosage form may be, e.g., 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, and 550 mg.

In certain embodiments, the 1-phenylalkanecarboxylic acid is administered orally and the additional neuroprotective agent(s) is administered separately, via the same or different route of administration. In certain embodiments, it may be necessary to administer the combination therapy separately due to incompatibility of the active agents, or due to inability of certain of the neuroprotective agents to be administered orally.

In certain embodiments, the pharmaceutical composition in accordance with the present invention may comprise an active agent comprising a therapeutically effective amount of a 1-phenylalkanecarboxylic acid and one or more of the following: Aβ peptides level reducers, pathogenic level tau reducers, microtubule stabilizers, agents capable or removing atherosclerotic plaques, agents that lower circulating levels of β-amyloid and tau, modulators of autophagy, neurotransmitter level regulators, GABA(A) α5 receptors inhibitors, and additional agents that help maintain and/or restore cognitive function and functional deficits of AD, and/or slow down decline in cognitive functions and functional deficits in AD,

In certain embodiments, the pharmaceutical composition in accordance with the present invention may comprise an active agent comprising a therapeutically effective amount of a 1-phenylalkanecarboxylic acid and one or more of the following: (a) one or more antibody[ies] capable of selectively recognizing prefibrillar pathological or neurotoxic tau and precursors comprising at least two tau proteins, or fragments thereof, cross-linked to each other, directly or through a linker (e.g., B4M), at one or more cysteine residues, as described above, (b) one or more immunogenic peptide[s] comprising at least two tau proteins cross-linked to each other, either directly or through a linker (e.g., B4M) at one or more cysteine residues, as described above, (c) an antisenilin antibody that discriminates between an Aβ peptide and the β-amyloid protein precursor from which it is proteolytically derived one or more segment[s] of the immunogenic peptides, (d) one or more segments[s] of the above antibodies, and (e) isolated genes or cDNA sequences encoding the above antibodies, (f) mixtures of any the foregoing and (ii) one or more pharmaceutically acceptable excipients. In the preferred embodiments, at least one of the cross-links between the individual tau monomers is not a disulfide bridge between cysteines of the monomers.

The active agent may also include one or more antibodies which are free end-specific of Aβ peptides and/or one or more immunogens for these antibodies; and/or a plurality of antibodies which recognize and bind ΔTau and do not recognize and do not bind hTau40, and/or one or more immunogens for these antibodies.

The specific embodiments contemplated include pharmaceutical compositions comprising a 1-phenylalkanecarboxylic acid together with, for example with one or more of the neuroprotective agents described above.

The embodiments contemplated also include uses of a conjugate of a cytoprotective agent (e.g., an antioxidant (e.g., melatonin or tocopherol) or an agent which will facilitate and/or improve antibody's ability to cross the blood brain barrier (BBB) (e.g., a hydrophobic substance which is capable of crossing the BBB, and is generally recognized as sage (GRAS) by the United States Food and Drug Administration (“FDA”)) in the pharmaceutical compositions and methods of the present invention.

The active agent(s) will generally comprise from about 0.01% to about 90% of the formulation, and the one or more excipients will generally comprise from about 10% to about 99.99% of the formulation. In the preferred embodiments, the formulations are used for introduction of the active agent into a body of a living mammal (e.g., a human) and are accompanied with instructions (e.g., a package insert) which recite directions for administration of the active agent into the body of the living mammal. In some of these embodiments, the formulations are used for treatment or prevention of AD and/or another tauopathy and are accompanied by the instructions which recited directions for treatment and/or prevention of AD and/or another tauopathy.

Pharmaceutical compositions of the present invention, in certain embodiments, may comprise a gene encoding an antibody capable of selectively recognizing pathogenic tau dimers and prefibrillar pathological or neurotoxic tau. Antibodies capable of selectively recognizing pathogenic tau dimers and prefibrillar pathological or neurotoxic tau were described above.

Pharmaceutical compositions in accordance with the present invention can be administered by parenteral, topical, intranasal, intravenous, oral, subcutaneous, intraarterial, intracranial, intraperitoneal, intranasal, or intramuscular means for prophylactic and/or therapeutic treatment. The pharmaceutical compositions can be administered intravenously, intracerebrally, intranasally, orally, transdermally, buccally, intra-arterially, intracranially, or intracephalically. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where deposits have accumulated, for example intracranial injection. For compositions comprising antibodies, intramuscular injection or an intravenous infusion may be preferred. A preferred route of administration for certain antibodies (e.g., camelid antibodies) may be oral. In some methods, particular therapeutic antibodies are injected directly into the cranium. In some methods, antibodies are administered as a sustained release composition or device, such as a Medipad™ device (Elan Pharm. Technologies, Dublin, Ireland). In certain embodiments, the adjuvant is alum.

The pharmaceutical formulations in accordance with the present invention may also contain one or more pharmaceutical carriers and/or suitable adjuvants.

A therapeutically effective amounts of 1-phenylalkanecarboxylic acid and one or more neuroprotective agent(s) used in the methods of treatment and pharmaceutical compositions of the present invention may vary according to factors such as the disease state, age, sex, and weight of the individual, the stage of the progression of the disease, and the ability of the modulator to elicit a desired response in the individual. Dosage regimens may be adjusted to provide the optimum therapeutic response. A therapeutically effective amount is also one in which any toxic or detrimental effects of the therapeutic agents are outweighed by the therapeutically beneficial effects.

A “prophylactically effective amount” refers to an amount effective, at dosages and for periods of time necessary, to achieve the desired prophylactic result, such as preventing or inhibiting the rate of Aβ formation, Aβ aggregation, tau deposition, tau aggregation, polymerization and/or neurotoxicity, and selective modulation of microglial activity in a subject predisposed to the formation of neurofibrillary tangles or AD. Typically, since a prophylactic dose is used in subjects prior to or at an earlier stage of disease, the prophylactically effective amount will be less than the therapeutically effective amount.

A “therapeutically effective amount” refers to an amount effective, at dosages and for periods of time necessary, to achieve the desired therapeutic rest, such as slowed progression of Alzheimer's disease, delayed onset, reduction or reversal of aggregate formation and/or neurofibrillary tangles, reduction or reversal of neurotoxicity, or selective modulation of microglial activity. A therapeutically effective amount of the neuroprotective agent of the invention may vary according to factors such as the disease state, age, sex, and weight of the individual, and the ability of the neuroprotective agent to elicit a desired response in the individual. Dosage regimens may be adjusted to provide the optimum therapeutic response. A therapeutically effective amount is also one in which any toxic or detrimental effects of the modulator are outweighed by the therapeutically beneficial effects.

Factors that may be considered when determining a therapeutically or prophylactically effective amounts of 1-phenylalkanecarboxylic acid and one or more neuroprotective agent(s) used in the methods of treatment of the present invention may, e.g., include concentration of Aβ peptides, tau, TNF-α, IL-1β, tau dimers, lipoproteins in a biological compartment of a subject, such as in the cerebrospinal fluid (CSF) or the plasma of the subject. It is to be noted that dosage values may vary with the severity of the condition to be alleviated. It is to be further understood that for any particular subject, specific dosage regimens could be adjusted over time according to the individual need and the professional judgment of the person administering or supervising the administration of the compositions. For example, a single bolus may be administered, several divided doses may be administered over time or the dose may be proportionally reduced or increased as indicated by the exigencies of the therapeutic situation.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration. Preferably, the carrier can be suitable for intravenous, intraperitoneal or intramuscular administration. Alternatively, the carrier is suitable for administration into the central nervous system (e.g., intraspinally or intracerebrally). In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Formulations for intravenous or intrathecal administration prepared in accordance with the present invention typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, a polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the 1-phenylalcanecarboxylic acids and the neuroprotective agents can be administered in a time-release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g., antibody to the prefibrillar pathogenic tau in the required amount) in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient plus any additional desired ingredient from a previously sterile-filtered solution thereof.

Topical application can result from transdermal or intradermal application. Topical administration can be facilitated by co-administration of the agent with cholera toxin or detoxified derivatives or subunits thereof. Alternatively, transdermal delivery can be achieved using skin patch or using transfersomes.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acids polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating conditions characterized by aggregates of amyloid beta peptides by placing the implant near portions of the brain affected by such aggregates, thereby effecting localized, high doses of the compounds of the invention.

Immunogenic agents of the present invention, such as peptides, may be administered in combination with an adjuvant. A variety of adjuvants can be used in combination with a peptide, such as tau, to elicit an immune response. Preferred adjuvants augment the intrinsic response to an immunogen without causing conformational changes in the immunogen that affect the qualitative form of the response.

A preferred class of adjuvants is aluminum salts (alum), such as aluminum hydroxide, aluminum phosphate, and aluminum sulfate. Such adjuvants can be used with or without other specific immunostimulating agents, such as 3 De-O-acylated monophosphoryl lipid A (MPL) or 3-DMP, polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn- -glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include (a) MF59 (WO 90/14837 to Van Nest et al., which is hereby incorporated by reference in its entirety), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi ImmunoChem, Hamilton, Mont.) containing 2% squalene, 0.2% Tween 80, and one or more bacterial c ell wall components from the group consisting of monophosphoryllipid A (MPL), trehalose dimycolate (TDM), and cell wall skeleton (CWS), preferably MPL+CWS (Detox™). Other adjuvants include Complete Freund's Adjuvant (CFA) and Incomplete Freund's Adjuvant (IFA). Other adjuvants include cytokines, such as interleukins (IL-1, IL-2, and IL-12), macrophage colony stimulating factor (M-CSF), and tumor necrosis factor (TNF). In certain embodiments, the adjuvant is ilum.

An adjuvant can be administered with an immunogen as a single composition, or can be administered before, concurrent with, or after administration of the immunogen. Immunogen and adjuvant can be packaged and supplied in the same vial or can be packaged in separate vials and mixed before use. Immunogen and adjuvant are typically packaged with a label, indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. However, alum, MPL or Incomplete Freund's adjuvant (Chang et al., Advanced Drug Delivery Reviews 32:173-186 (1998), which is hereby incorporated by reference in its entirety) alone or optionally all combinations thereof are suitable for human administration.

Agents of the present invention are often administered as pharmaceutical compositions comprising an active therapeutic agent and a variety of other pharmaceutically acceptable components. See Remington's Pharmaceutical Science (15th ed., Mack Publishing Company, Easton, Pa., 1980), which is hereby incorporated by reference in its entirety. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

Antibody-drug conjugates (ADCs) combine the binding specificity of (monoclonal) antibodies with the potency of chemotherapeutic agents. In certain preferred embodiments of the invention, the pharmaceutical composition comprises a conjugate of the a 1-phenylalkanecarboxylic acid together with an antibody as described herein, e.g., an antibody capable of selectively recognizing a free N-terminus of an amyloid β-peptide or a free C-terminus of amyloid β-peptide Aβ1-40 or an antibody capable of selectively recognizing a neurotoxic tau or a precursor of a neurotoxic tau. Conjugation of drugs to antibodies, either directly or via linkers, involves a consideration of a variety of factors, including the identity and location of the chemical group for conjugation of the drug, the mechanism of drug release, the structural elements providing drug release, and the structural modification to the released free drug. Antibody-drug conjugates (ADCs) are known to those having ordinary skill in the art and may utilize, for example, a linker between the antibody and drug such as that described in U.S. Pat. No. 8,586,049 (which is incorporated herein by reference in its entirety) (a linker unit selected from the group consisting of maleimidocaproyl and maleimidocaproyl-Val-Cit-PABA), or drug linker compounds as described in U.S. Pat. No. 8,609,105 (which is incorporated herein by reference in its entirety) represented by the general formula: D-LU (I) or a pharmaceutically acceptable salt or solvate thereof, wherein LU is a Linker unit and D (in that case) is an auristatin having a C-terminal carboxyl group that forms an amide bond with the linker unit which comprises at least one amino acid. Such antibody-drug conjugates may be administered in any form which provides efficacy to the (human) patient, including but not limited to oral or parenteral formulations).

In yet other embodiments of the present invention wherein the 1-phenylalkanecarboxylic acid compound and at least one additional neuroprotective agent are incompatible when in contact with each other (e.g., causing one or both of the agents to be rendered unstable or degraded), the agents may be separated in an oral dosage form via the use of a bilayer tablet or a capsule within a capsule.

For example, in the case of a bilayer tablet, the 1-phenylalkanecarboxylic acid compound may be present in a first layer and the additional neuroprotective agent(s) is present in a second layer, wherein the layers are in direct physical contact and at least one binder is present in the first layer and/or the second layer. Such a pharmaceutical composition is preferably formulated for immediate release of both active agents.

On the other hand, the 1-phenylalkanecarboxylic acid compound may be present in a first capsule and the additional neuroprotective agent(s) is present in a second capsule, wherein one of the capsules is contained within the other capsule. Such arrangements are known in the art and described, e.g., in U.S. Pat. No. 7,670,612, which is incorporated herein by reference in its entirety.

For parenteral administration, agents of the present invention can be administered as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention, particularly, antibodies, can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entireties).

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391:851 (1998), which is hereby incorporated by reference in its entirety). Co-administration can be achieved by using the components as a mixture or as linked molecules obtained by chemical crosslinking or expression as a fusion protein. Alternatively, transdermal delivery can be achieved using a skin path or using transferosomes (Paul et al., Eur. J. Immunol. 25:3521-24 (1995); Cevc et al., Biochem. Biophys. Acta 1368:201-15 (1998), which are hereby incorporated by reference in their entireties).

### Vaccines

The additional neuroprotective agent(s) administered in combination with the 1-phenylalkanecarboxylic acid may be administered as a vaccine in order to provide passive immunization and/or active immunization to a mammal.

A vaccine for active or passive immunization may comprise one or more antibody[ies] which are free end-specific of Aβ peptides and/or one or more immunogens for these antibodies, or which are capable of selectively recognizing prefibrillar pathological or neurotoxic tau and/or precursors comprising at least two tau proteins, or fragments thereof, cross-linked to each other, directly or through a linker (e.g., B4M), at one or more cysteine residues including their pathogenic conformation. In certain embodiments, at least one of the cross-links between the individual tau monomers is not a disulfide bridge between cysteines of the monomers.

The vaccine for active immunization may also comprise one or more epitopes of antibody[ies] which are free end-specific of AO peptides and/or one or more immunogens for these antibodies and/or of the antibody[ies] capable of selectively recognizing prefibrillar pathological or neurotoxic tau and precursors, including their pathogenic conformation.

The neuroprotective agents suitable for inclusion into vaccines of the invention were described in detail above.

Any one of these vaccines may include also one or more antibodies which are free end-specific of Aβ peptides and/or one or more immunogens for these antibodies; and/or a plurality of antibodies which recognize and bind ΔTau and do not recognize and do not bind htau1-40, and/or one or more immunogens for these antibodies. Some of the embodiments contemplated were described above the Pharmaceutical Composition section.

The vaccine may also additionally comprise one or more pharmaceutically acceptable excipients as described above and, in certain embodiments, one or more mimotopes of any of the antibodies mentioned above, and may be administered as described above (e.g., intravenously, subcutaneously, intranasally or intracranially).

### Therapy

The pharmaceutical compositions of the present invention can be used as a therapy to treat proteinopathies such as Alzheimer's disease, or a tauopathy associated with the development of neurofibrillary tangles. Additionally, the administration of these substances and compositions can also be used as a prophylactic treatment to immunize against Alzheimer's disease, or the tauopathy associated with the development of neurofibrillary tangles.

Patients amenable to treatment include individuals at risk of disease but not showing symptoms, as well as patients presently showing symptoms. In the case of Alzheimer's disease, virtually anyone is at risk of suffering from Alzheimer's disease. Therefore, the present methods can be administered prophylactically to the general population without the need for any assessment of the risk of the subject patient. Such prophylactic administration can begin at, e.g., age 50 or greater. The present methods are especially useful for individuals who do have a known genetic risk of Alzheimer's disease. Such individuals include those having relatives who have experienced this disease and those whose risk is determined by analysis of genetic or biochemical markers. Genetic markers of risk toward Alzheimer's disease include mutations in the APP gene, particularly mutations, at position 717 and positions 670 and 671 referred to as the Hardy and Swedish mutations respectively. Other markers of risk are mutations in the presenilin genes, PS1 and PS2, and ApoE4, family history of AD, hypercholesterolemia or atherosclerosis. Individuals presently suffering from Alzheimer's disease can be recognized from characteristic dementia by the presence of risk factors described above. In addition, a number of diagnostic tests are available for identifying individuals who have AD. These include imaging, and/or measurement of CSF tau and Aβ42 levels. Elevated tau and decreased Aβ42 levels signify the presence of AD. Individuals suffering from Alzheimer's disease can also be diagnosed by Alzheimer's Disease and Related Disorders Association criteria.

In asymptomatic patients, treatment can begin at any age (e.g., 10, 20, 30, 40, 50, or 60). Usually, however, it is not necessary to begin treatment until a patient reaches 40, 50, 60, 70, 75 or 80. Treatment typically entails multiple dosages over a period of time. Treatment can be monitored by assaying lipoprotein levels, Aβ peptide levels, tau levels, TNF-α levels, IL-1β levels, antibody levels, or activated T-cell or B-cell responses to the therapeutic agent over time. If the response falls, a booster dosage is indicated. In the case of potential Down's syndrome patients, treatment can begin antenatally by administering therapeutic agent to the mother or shortly after birth.

In prophylactic applications, pharmaceutical compositions or medicaments are administered to a patient susceptible to, or otherwise at risk of, Alzheimer's disease in an amount sufficient to eliminate or reduce the risk, lessen the severity, or delay the outset of the disease, including biochemical, histological and/or behavioral symptoms of the disease, its complications and intermediate pathological phenotypes presented during development of the disease. In therapeutic applications, compositions or medicaments are administered to a patient suspected of, or already suffering from, such a disease in an amount sufficient to cure, or at least partially arrest, the symptoms of the disease biochemical, histological and/or behavioral), including its complications and intermediate pathological phenotypes in development of the disease. In some methods, administration of agent reduces or eliminates mild cognitive impairment in patients that have not yet developed characteristic Alzheimer's pathology. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, the immune response is monitored and repeated dosages are given if the immune response starts to wane.

Effective doses of the compositions of the present invention, for the treatment of the above described conditions vary depending upon many different factors, including means of administration, target site, physiological state of the patient, other medications administered, and whether treatment is prophylactic or therapeutic. Treatment dosages need to be titrated to optimize safety and efficacy.

An additional advantage of the selective antibodies of the present invention, in certain embodiments, may be that, for equal mass dosages, dosages of antibodies that selectively recognizing the conformation of the prefibrillar pathological or neurotoxic tau oligomers and their precursors (i.e., tau dimers) comprising at least two tau proteins, or fragments thereof, cross-linked to each other, directly or through a linker (e.g., B4M), at one or more cysteine residues contain a higher molar dosage of the antibodies effective in clearing and/or “inactivating,” than a composition comprising a mixture of the selective antibodies and non-selective antibodies.

The amount of immunogen depends on whether adjuvant is also administered, with higher dosages being required in the absence of adjuvant. Generally, the amount of an immunogen for administration sometimes varies from 1 to 500 μg per patient and more usually from 5 to 500 μg per injection for human administration. Occasionally, a higher dose of 1 to 2 mg per injection is used. Typically about 10, 20, 50, or 100 μg is used for each human injection. The mass of immunogen also depends on the mass ratio of immunogenic epitope within the immunogen to the mass of immunogen as a whole. Typically, 10⁻³ to 10⁻⁵ micromoles of immunogenic epitope are used for each microgram of immunogen. The timing of injections can vary significantly from once a day, to once a year, to once a decade. On any given day that a dosage of immunogen is given, the dosage is greater than 1 μg/patient and usually greater than 10 μg patient if adjuvant is also administered, and greater than 10 μg/patient and usually greater than 100 μg/patient in the absence of adjuvant. A typical regimen consists of an immunization followed by booster injections at time intervals, such as 6 week intervals. Another regimen consists of an immunization followed by booster injections 1, 2, and 12 months later. Another regimen entails an injection every two months for life. Alternatively, booster injections can be on an irregular basis as indicated by monitoring of immune response.

For passive immunization with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1 to 10 mg/kg. An exemplary treatment regime entails administration once per every two weeks or once a month or once every 3 to 6 months. In some methods, two or more antibodies (e.g., recombinant, monoclonal, chimeric and/or humanized) with the same or different binding specificities are administered simultaneously, in which case the dosage of each antibody administered falls within the ranges indicated. In such circumstances, the two or more antibodies may both be directed at, e.g., truncated tau. Alternatively, one or more of the antibodies may be directed at, e.g., truncated tau, and one or more additional antibodies may be directed at amyloid-β (Aβ) peptides associated with Alzheimer's disease. Antibodies are usually administered on multiple occasions. Intervals between single dosages can be hourly, daily, weekly, monthly, or yearly. In some methods, dosage is adjusted to achieve a plasma antibody concentration of 1 to 1000 μg/ml and in some methods 25-300 μg ml. Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Doses for nucleic acids encoding immunogens range from about 10 ng to 1 g, 100 ng to 100 mg, 1 μg to 10 mg, or 30-300 μg DNA per patient. Doses for infectious viral vectors vary from 10 to 100, or more, virions per dose.

In certain embodiments, the efficacy of the administration/treatment may be accessed by measuring levels of neurotoxic tau in plasma and/or CSF. Based on this assessment, the dose and/or frequency of administration may be adjusted accordingly. In addition or in alternative, the efficacy of administration/treatment is accessed by, e.g., monitoring the number of NFTs.

In addition or in alternative, the efficacy of the administration/treatment may also be accessed by amyloid plaques imaging by PET. An increase in brain's metabolism would indicate that the administration/treatment is effective. The efficacy may further be accessed by a degree of brain atrophy, as determined by MRI.

In addition or in alternative, the efficacy of the administration/treatment may be accessed by measuring the levels of IgG and IgM against dimer of tau or oligomers of tau.

The safety of the administration/treatment may be accessed by monitoring for microhemorrhages and/vasogenic edema, e.g., by MRI. Based on this assessment, the dose and/or frequency of administration may be adjusted accordingly.

Antibodies and immunogens may be administered intranasally, by a subcutaneous injection, intramuscular injection, IV infusion, transcutaneously, buccally, etc., alone or in combination with other immunological therapeutic agent(s) for the treatment of tauopathies (e.g., AD).

Other features of the invention will become apparent in the course of the following descriptions of exemplary embodiments which are given for illustration of the invention and are not intended to be limiting thereof.

### EXAMPLES

### Example 1

In Example 1, the safety and tolerability and cognitive effects of CHF 5074 was analyzed after prolonged treatment in MCI (mild cognitively impaired) patients.

Methods: At the end of a 14-week double-blind, placebo-controlled study in 96 MCI patients evaluating three titrated dose regimens of CHF 5074 (200, 400 and 600 mg/day), patients were given the option to enter a 76-week open label extension study. Patients received CHF 5074 at the dose equal to that of their originally assigned double-blind study cohort. Patients were monitored for vital signs, cardiac activity, neuropsychological performance and safety laboratory parameters.

Results: Seventy-four patients entered the open label study: 26, 21 and 27 in the 200,400 and 600 mg/day cohorts, respectively. At Study Week 40, 14 patients dropped out: 4, 2 and 8 in the 200,400 and 600 mg/day cohorts, respectively. Three of drop-outs were for adverse events: two in the 600 mg/day group (serum creatinine elevation and worsening of cognitive function) and one in the 400 mg/day group (pneumonia). The most frequent treatment-emergent adverse events were gastrointestinal disorders, with diarrhea being reported by 1.4% of patients on 200 mg/day, 6.3% of patients on 400 mg/day and 16.0% of patients on 600 mg/day. Interim analysis of cognitive tests of 32 patients reaching Study Week 64 showed statistically significant improvements compared to Baseline on Digit Symbol Substitution Test (+4.8±1.1 matches, p<0.001), Trail Making Test-A (−8.1±2.4 sec, p=0.002), Trail Making Test-B (−14.5±4.6 sec, p=0.004), Immediate Word Recall (+2.9±0.8 words, p=0.001) and Delayed Word Recall (+1.3±0.4 words, p=0.003). APOE4 carriers performed significantly better than APOE4 non-carriers on Immediate Word Recall (+5.4±1.2 vs+1.4±0.9 words, p=0.012) and Trail Making Test-A (−12.4±2.8 vs −5.6±3.3 sec, p=0.034) with improvements representing 25-38% of Baseline scores.

CHF 5074 was well tolerated by MCI patients after prolonged treatment at doses up to and including 400 mg/day. Drug treatment was associated with sustained cognitive benefit in executive function and verbal memory for at least 64 weeks.

At the end of this study, the following were made: CHF 5074 dose-dependently lowered neuroinflammation biomarkers in MCI patients; CHF 5074 demonstrated an acceptable safety profile in MCI patents; CHF 5074 treatment was associated with sustained cognitive benefit in verbal memory and executive function for at least 88 weeks; and the results justify the conduct of Phase 3 studies in amnestic MCI ApoE4 carriers and in asymptomatic ApoE4 carriers with parental history of AD.

### Example 2

### Aim of the Study

With the goal of optimizing the neuroprotective activity of CHF 5074, the association of CHF 5074 with resveratrol, a SIRT1 activator, will be studied. Resveratrol is a widely studied polyphenol endowed with anti-aging, anti-inflammatory and anti-oxidant properties (Yu W, Fu Y C, Wang W. Cellular and molecular effects of resveratrol in health and disease. J Cell Biochem 2012; 113: 752-759 (which is incorporated herein by reference in its entirety)). Resveratrol acts mainly through major activation of sirtuin 1 (Howitz K T, Bitterman K J, Cohen H Y, Lamming D W, Lavu S, Wood J G, et al. Small molecule activators of Sirtuins extend Saccharomyces cerevisiae lifespan. Nature 2003; 425: 191-196(which is incorporated herein by reference in its entirety)).

### Neuronal Cultures

Primary cultures of mouse cortical neurons C57BL/6 mice are purchased from Charles River Italia. Primary cortical neurons will be prepared from cortices of 15-day embryonic mice and cultured as previously described (Sarnico I, Lanzillotta A, Boroni F, Benarese M, Alghisi M, Schwaninger M, et al. NF-kappaB p50/RelA and c-Rel-containing dimers: opposite regulators of neuron vulnerability to ischaemia. J Neurochem 2009; 108: 475-485(which is incorporated herein by reference in its entirety)). Cells are plated at a density of 1.0×105 cells/cm2 in 2 cm² culture dishes for the viability studies, in 21 cm² culture dishes for Western blot and co-immunoprecipitation analyses. Experiments will be carried out at 11 days in vitro (DIV).

### Oxygen Glucose Deprivation

Oxygen glucose deprivation (OGD) is performed in cortical neurons for 3 h as previously described (Sarnico et al, 2009 (which is incorporated herein by reference in its entirety)). Control cell cultures are incubated in a normal aerated incubator for the same time period. At the end of the OGD period, cells are transferred to recover in Neurobasal medium containing 0.4% B27 supplement with or without CHF 5074 (1, 3 or 10 mM) resveratrol (1, 3 or 30 μM) alone or in combination. Resveratrol (Merck Chemicals Limited, UK) is dissolved in dimethyl sulfoxide (DMSO) and diluted before application to a final DMSO concentration lower than 0.3%. The cell viability is estimated 24 h later.

Extraction of cell proteins is performed 2 h after the OGD period. Neuronal injuries are evaluated by measuring the amount of lactate dehydrogenase (LDH) released into the culture medium relative to total releasable LDH, using the CytoTox 96® Non-Radioactive Cytotoxicity Assay (Promega Corporation, Wisconsin, USA).

### Immunocitochemistry

After exposure to 3 h OGD and 24 h reoxygenation, cortical neurons are fixed for 15 min by Immunofix (Bio-Optica, Italy). Cells are incubated for 15 min with 0.2% Igepal (Sigma-Aldrich) and 0.3% H2O2 in 0.1 M PBS to inhibit endogenous peroxidases; then blocked 1 h in 0.1 M PBS containing 3% BSA (Sigma-Aldrich, Italy) and 0.2% Igepal. Neurons are incubated for 2 h at 37° C. with rabbit polyclonal anti-cleaved caspase-3 (c-casp-3) antibody (R&D AF 835) in 0.1 M PBS containing 3% BSA and 0.2% Igepal. Primary antibody is detected by biotinylated anti-rabbit secondary antibody in PBS 0.1 M and 1% BSA, incubated 1 h in the dark. The signal is revealed by incubation for 45 min in the dark with AB Complex (Vector PK-4000), visualized with 3,3′-diaminobenzidine (Sigma-Aldrich, Italy) and 1% H2O2in 0.1 M PBS. The cells are subsequently counter-stained with hematoxylin, dehydrated in ethanol, and mounted with DPX upon slides. Quantification of cell apoptosis is performed by countingc-casp-3-positive cells and hematoxylin stained neurons and data are expressed as percentage of c-casp-3-positive cells to total cell number. Terminal deoxynucleotidyl transferase-mediated dUTP-nick-end labeling (TUNEL) is performed using the kit purchased by Roche Molecular Biochemicals (Indianapolis, Ind., USA) according to the manufacturer's instructions.

### Example 3

Aim of the Study

The effects of long-term treatment with CHF 5074, MABT5102A (Crenezumab) and their combination on brain pathology and memory deficits will be evaluated in a transgenic mouse model of AD (Tg2576 mice). It has been shown that MABT5102A binds to soluble, oligomeric and fibrillar β-amyloid deposits (Adolfsson 0, Pihlgren M, Toni N, Varisco Y, Buccarello A L, Antoniello K, et al., An effector-reduced anti-β-amyloid (Aβ) antibody with unique Aβ binding properties promotes neuroprotection and glial engulfment of Aβ. J Neurosci 2012; 32: 9677-89, which is incorporated herein by reference in its entirety). Crenezumab has an IgG4 backbone which reduces effector function.

### Animals and Treatments

Mice over-expressing the human APP gene carrying the Swedish double mutation (K670N/M671L) under the transcriptional control of the hamster prion protein promoter are used (Tg2576 mice). Only female mice will be included in the experimental groups. Animals of 6-month of age are assigned to chronic treatment with CHF 5074 (375 ppm in the diet, equivalent to approximately 60 mg/Kg/day; n=15) or MABT5102A (10 mg/kg s.c. once weekly; n=15), CHF 5074+MABT5102A (n=15), vehicle (standard diet+saline s.c. once weekly, n=15) for 9 months. Groups are balanced for gender and body weight. Ten non-transgenic, wild-type mice B6/SJL strain are included in the study. They are housed and handled starting from 6 months of age up to 15 months of age in the same condition as the transgenic animals. Body weight and food consumption are monitored once a week. Animals are regularly checked for spontaneous or stimulated locomotor activity. Genotyping of Tg2576 mice is performed at the beginning of experiment to confirm the presence of the human APP gene. At the end of treatment, blood samples are collected in EDTA-coated tubes and centrifuged at 800 g for 20 min to separate serum. Serum samples are divided into two aliquots of approximately 100 mL each and stored at −80° C. Tissue samples of liver, kidneys, spleen, esophagus, stomach, duodenum, jejunum, ileum, cecum, colon, rectum and hemopoietic tissue, fixed in 10% buffered formalin, are trimmed, dehydrated, embedded in paraffin wax and sectioned at 5 mm thickness. Slides are stained with hematoxylin and eosin and examined by a blinded skilled pathologist for the qualitative evaluation of any treatment related changes.

### Behavior

On the last 3 days of treatment, long-term memory is evaluated in the novel object recognition task, which measures recognition memory under spontaneous behavioural conditions. Mice are tested in an open-square grey arena (40×40 cm), 30 cm high, with the floor divided into 25 squares. A black plastic cylinder, a glass vial and a metal cube, made in copy of three, are used as objects of choice for the test, based on previous verification that they are all equally investigated with no bias in their saliency. Object presentation is carefully randomized across the animals, which are observed through a Noldus videocamera positioned above the apparatus during the experiments. The task starts with a habituation trial during which the animals are placed in the empty arena for 5 min and their movements manually recorded as the number of square-crossings, in order to evaluate mouse exploratory and motor behaviour. The next day, mice are placed in the same arena containing two identical objects (familiarization phase). Exploration is manually recorded in a 10-min trial by an investigator blinded to the strain and treatment. Left and right familiar objects are recorded separately in order to evidence eventual side preference, whereas the calculation of the total investigation time on both objects allows analyzing mouse exploratory behavior on the objects during training. Sniffing, touching and stretching the head toward the object at a distance not more than 2 cm are scored as object investigation. Twenty-four hours later (test phase) mice are again placed in the arena containing two objects: one identical to one of the objects presented during the familiarization phase (familiar object), and a new one (novel object), and the time spent exploring the two objects is recorded for 10 min. Memory is expressed as a discrimination index, i.e. (seconds on novel−seconds on familiar)/(total time on objects). Animals with no memory impairment spend longer investigating the novel object, giving a higher discrimination index.

### Brain Morphology

Two-days after the behavioral testing, all animals are deeply anesthetized and killed by decapitation for tissue sampling. Tissues of interest are rapidly dissected out and half of the brain will be fixed in 4% paraformaldehyde in 0.1 M Sorensen phosphate buffer, pH 7.0 for 24 h, then rinsed for at least 48 h in 5% sucrose in 0.1 M phosphate buffer. Brains are frozen in CO2 and 14 mm thick coronal sections are then obtained from the dorsal hippocampus (bregma—3.30 mm level according to Paxinos & Watson, 1998) using a cryostat (Kriostat 1750, Leitz, Germany) and collected on gelatin coated slides. The following primary antisera are used: goat anti-doublecortin (Doublecortin C-18, 1:150 dilution, Santa Cruz Biotechnology Inc, Heidelberg, Germany); mouse anti-synaptophysin antibodies (clone SY38, MAB5258, 1:1000 dilution, Millipore, Billerica, Mass.); rabbit anti GFAP antibody (1:300 dilution, Euro-Diagnostics Resources, Apeldoorn, The Netherlands); rat anti-mouse CD11b monoclonal antibody (1:50 dilution, Chemicon International, Temecula, Calif.). Doublecortin-, synaptophysin- and GFAP-immunoreactivity is detected by indirect immunofluorescence; microglia by ABC histochemistry. For immunofluorescence experiments, sections are first incubated in 0.1 M phosphate buffered saline (PBS) at room temperature, followed by incubation at 4° C. for 24 h in a humid atmosphere with the primary antibodies diluted in PBS containing 0.3% Triton X-100, v/v. After rinsing in PBS, the sections are incubated at 37° C. for 30 min in a humid atmosphere with the secondary antisera conjugated with different fluorochromes (CyTM2—and Rhodamine Red™-X-conjugated AffiniPure donkey anti-rabbit, anti-mouse, anti-goat, Jackson Immunoresearch, West Grove, Pa.) diluted in PBS containing 0.3% Triton X-100. Sections are then rinsed in PBS and mounted in a mixture of PBS and glycerol-containing paraphenylenediamine (Sigma). Images from tissues are taken by Olympus AX70-Provis and Nikon Eclipse 600 microscope equipped with motorized z-stage control and F-view II CCD Cameras. Immunofluorescence staining is analyzed using the Image ProPlus software (Media Cybernetics Inc, Bethesda, Md.). Analysis of all indicated markers is carried out on three non-consecutive sections per animal. The number of doublecortin-immunoreactive cells is counted and normalized for the dental gyrus length (1500 mm). Analysis of synaptophysin optical density is executed in the areas 1 and 2 the parietal cortex on three non-consecutive sections on original images with intensity values corresponding to the grey scale of image. Twenty× magnification images are captured using a rectangular frame. After setting a threshold to minimize background, the mean optical density of pixels is computed based on a scale of 0-256 relative units. Background values are taken from a white-matter structure (corpus callosum) and subtracted from the mean optical density of grey level. Immunoreactivity for GFAP-positive cells (percent area fraction) is measured around “large” (diameter >55 micron) Ab plaques located in the cerebral cortex (stained with 6E10 antibodies), using a sampling frame formed by concentric rings, starting from the centre to the border of the plaque. Immunoreactivity is detected using a threshold procedure (Image ProPlus) and the percentage of immunoreactive area will be measured in each ring collocated around the plaque.

### Brain β-Amyloid Levels

One frozen brain hemisphere is weighed and mechanically homogenized (1 mL syringe, gauge 20 needle, 10 repeats) in 5 vol/weight of TBS (Tris HCl 50 mM pH7.6; NaCl 150 mM; EDTA 2 mM) containing protease inhibitors (Complete™, Roche, Basel, Switzerland). Homogenate is aliquoted and stored at −80° C. for the measurement of sodium dodecyl sulphate (SDS), and formic acid (FA)-soluble Aβ40 and Aβ42. One additional aliquot is dedicated to measurement of oligomeric AP. For SDS-soluble and FA-soluble Aβ assessments, one aliquot of each sample is suspended in 2% SDS containing protease inhibitors (2×, Roche's Complete Protease Inhibitor Cocktail Tablets) and centrifuged at 16,000×g for 10 min. Supernatant is collected (1st SDS aliquot), pellet is washed by re-suspension in 2% SDS containing protease inhibitors and thereafter re-centrifuged at 100,000×g for 1 hour. Supernatant is collected (2nd SDS aliquot) and stored at −80° C. until assay. The remaining pellet is extracted using 70% FA in water and centrifuged at 100,000×g for 1 hour. Supernatant is collected and stored at −80° C. until assay. The levels of Aβ40 and Aβ42 in all samples are determined employing the commercially available ELISA kits purchased from Innogenetics. Data obtained in brain homogenates are expressed as pmoles/g wet weight tissue.

### Brain Aβ Oligomers

Brain Aβ oligomer levels are determined in subchronically-treated mice by immunoprecipitation/western blotting (IP/WB) using a modified version of a previously described procedure (Lesne S, Koh M T, Kotilinek L, Kayed R, Glabe C G, Yang A, Gallagher M, Ashe K H (2006). A specific amyloid-beta protein assembly in the brain impairs memory. Nature 440, 352-357, which is incorporated herein by reference in its entirety). Hemi-forebrain samples in 500 μL of a solution containing 50 mM Tris-HCl (pH 7.6), 0.01% NP-40, 150 mM NaCl, 2 mM EDTA, 0.1% SDS, 1 mM phenylmethylsulfonyl fluoride and a protease inhibitor cocktail (Sigma-Aldrich; P8340) are mechanically disaggregated by repeated passages (up to 5) through a syringe needle (gauge 20). The resulting samples are centrifuged at 3,000 rpm for 10 min at 4° C. and the supernatant (SN1) is further clarified by centrifugation at 14,000 rpm for 90 min at 4° C. The pellet (P1) is resuspended in 50 mM Tris-HCl (pH 7.6), 150 mM NaCl, 0.1% Triton X-100, disaggregated with a micropipettor (10 passages), and centrifuged at 14,000 rpm (90 min, 4° C.) to generate SN2. Supernatants 1 and 2, representative of the extracellular and the cytoplasmic fraction, respectively, are combined, followed by total protein determination (Bio-Rad Protein Assay Dye Reagent with bovine serum albumin as standard). Equal total protein amounts of each extract (500 mg brought to a final volume of 500 mL with phosphate-buffered saline, PBS) are directly used for Aβ oligomer analysis by IP/WB. To this end, extracts are first incubated for 2 h at 4° C. with 30 mL of Dynabeads Protein G (Invitrogen) to eliminate endogenous immunoglobulins and other polypeptides non-specifically binding to Protein G, followed by a further incubation of unbound polypeptides with 4 mg of the anti-Aβ monoclonal antibody (mAb) 4G8 for 18 h at 4° C. mAb 4G8, which recognizes amino acids 17-24 of the Aβ peptide, are chosen as immune-capture antibody because it does not react with sAPP-alpha (amino acids 18-687), and thus allows to eliminate interference by the latter polypeptide that is abundantly produced by Tg2576 mice. Immunoprecipitation is carried out by incubating with Dynabeads Protein G (40 mL) for 2 h at 4° C. in a rotary shaker. Following magnetic separation, the beads are washed with PBS, and fractions eluted by heating for 15 min at 70° C. in SDS-containing sample buffer, are electrophoresed on pre-cast 4-12% Bis-Tris Midi Gels in MES buffer (Invitrogen). Fractionated proteins are electro-transferred to 0.2 μm nitrocellulose membranes, which are boiled for 25 sec in PBS, and blocked with 5% bovine serum albumin in Tris-buffered saline prior to the addition of the anti-Aβ biotinylated mAb 6E10 (1:400) in a SnaP i.d. blotting system (Millipore). This is followed by the addition of IrDye 680 streptavidin (1:3000; LI-COR) and visualization of immune-reactive bands by near infrared fluorescence with an Odyssey (LI-COR) imager. Non-specific, mAβ 6E10 cross-reactive polypeptides, present in both wild-type and Tg2576 brain extracts, are used as loading controls and as internal references for data normalization. Synthetic Aβ42 (n) oligomers, with n-values ranging from 1 to 4, are used as set-up controls for IP/WB analysis.

### Intraneuronal APP/A13

Indirect immunofluorescence is used to determine intracellular Aβ. Briefly, animals are sacrificed, brains are rapidly removed, immersed in 4% paraformaldehyde for 24 hours, and then washed in 5% sucrose in phosphate buffer. Sections (14 mm thickness) are cut from layers II-III of the medial cortex with a cryostat. Intraneuronal APP/Aβ are stained with 6E10 anti-Aβ1-16 monoclonal antibody (Covance, Princeton, N.J.) and visualized with a Rhodamine Red-X-conjugated anti-mouse antiserum (Jackson ImmunoResearch, Baltimore, Pa.). This antibody reacts to the abnormally processed isoforms, as well as precursor forms. About 50 neurons are analyzed in anterior cingulated cortex in each animal; all sections are processed at the same time. Stained specimen is analyzed with a Nikon 600 Eclipse microscope, equipped with a Nikon DXM1200F digital camera (Nikon Italia, Florence, Italy). The ProPlus software (Media Cybernetics Inc, Bethesda, Md.) is used to evaluate optical density in single cells. The mean value over about 50 neurons/animal is used for statistical analysis.

### Brain β-Amyloid Plaques and Activated Microglia

Coronal sections range from bregma −1.46 mm (anterior) to −2.06 mm (posterior), according to Paxinos & Watson, 1998. Aβ plaques immunohistochemistry is performed using 1:250-diluted 6E10 monoclonal biotinylated antibody (Signet Laboratories, Dedham, Mass.) as primary antibody. Sections are first incubated in Tris-buffered saline pH7.6 (TBS) at room temperature for 10-30 min, followed by incubation in 3% H₂O₂ distilled water solution for 15 minutes. After rinsing in TBS for 10 minutes the sections are incubated at 4° C. overnight in a humid atmosphere with the primary antibodies diluted in TBS containing 0.3% Triton X-100. The antibody is prepared adding 6E10 4 μL to TBS 1000 μL and Blocking Reagent 40 μL. After rinsing in TBS for 10 min (2×5 min), the sections are incubated 60 min in a humid atmosphere with streptavidin-peroxidase solution, according to the mouse-on-mouse kit peroxidase procedure (Dako Cytomation, Glostrup, Denmark) as revealing system. After rinsing in TBS for 10 min, peroxidase activity is detected by treatment with 3,3′-diaminobenzidine (DAB) for 5 minutes. The sections are cleared and mounted with mounting medium in xylene for histology. Slides are photographed using a digital Nikon DS microscope color camera. Digital images are analyzed using NIS-Elements software (Nikon, Tokyo, Japan). Each image is analyzed using the automated target detection mode. Images sizes are 1280×960 pixels and target area will have a size of 68,000 mm². The software determines the numbers of plaques, mean areas of plaques, and plaque area fraction (immunopositive area/total area used as scan object). Twelve counts for each level of the three levels considered are performed. Analyses are carried out in analogous areas of the cortex and hippocampus using a 10× objective.

Activated microglia in CA1 region of hippocampus is immunodetected using 1:50 diluted CD11β rat anti-mouse monoclonal antibody. For the counts in this region, a 20× objective is used and a target area of 127,000 mm². Sections are first incubated in TBS at room temperature for 10-30 min, followed by incubation in 3% 11202 distilled water solution for 15 minutes. After rinsing in TBS for 10 minutes the sections are incubated with normal goat serum (1:20) diluted in TBS for 20 minutes. The excess serum is eliminated and the sections are incubated at 4° C. overnight in a humid atmosphere with the primary antibodies CD1113 (1:50) diluted in TBS containing 0.3% Triton X-100. After rinsing in TBS for 10 min (2×5 min), the sections are incubated 30 min in a humid atmosphere with biotinylated secondary antibody solution according to the Vectastain ABC Elite system (Vector, Sacramento, Calif.) as revealing system. After rinsing in TBS for 10 min, the sections are incubated with Vectastain ABC reagent for 30 minutes, followed by appropriate washing and peroxidase activity detection by treatment with DAB. The sections are cleared and mounted with mounting medium in xylene for histology (Carlo Erba, Milano, Italy).

### Brain Tau and Hyperphosphorylated Tau Levels

Tau and phospho-tau are analyzed by Western blotting in brain extracts from mice treated subchronically with CHF 5074-medicated or standard diet. Brain lysates (50 μg total protein each) are suspended in sample loading buffer and fractionated on 4-12% SDS/polyacrylamide gradient gels. Proteins are then transferred to nitrocellulose membranes, followed by immunodetection, incubating the membranes overnight (4° C.), with the following primary antibodies: phospho-tau PHF1 mouse antibody (1:100) and phospho-tau CP13 antibody (1:100); anti-tau mouse antibody (1:3000, Cell Signaling Technology, Danvers, Mass., USA), and anti-βIII tubulin antibody (1:1000 Sigma-Aldrich, Sigma, St. Louis, Mo., USA). Immunoreactions are revealed by 1 h incubation at 37° C. with secondary antibody coupled to horseradish peroxidase (1:1500) (Santa Cruz Biotechnology, CA, USA), followed by chemoluminescence detection using ECL Western blotting reagents (GE Healthcare). Immunoblot quantification is performed by densitometric scanning, using the GelPro Analyser software (Media Cybernetics, Bethesda, Mo., USA). Data are expressed as the ratio of tau and phospho tau forms relative to bIII-tubulin.

In an analogous way, the combinations of CHF 5074 with the compounds MK-8931, PTB2, indole-3-propionic acid and pioglitazone are tested.

### Example 4

The composition of an exemplary formulation in form of tablets is reported in Table 1.

TABLE 1

| Ingredients | Quantity (mg) |
|---|---|
| CHF 5074 | 100 |
| Resveratrol | 250 |
| Lactose monohydrate | 85 |
| Microcrystalline cellulose | 45 |
| Sodium lauryl sulfate | 20 |
| Total amount | 500 |

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A pharmaceutical composition, comprising from 50 mg to 550 mg of CHF 5074 together with at least one pharmaceutical excipient.

2. The pharmaceutical composition of claim 1, wherein the composition is suitable for oral administration.

3. The pharmaceutical composition of claim 1, comprising from 200 mg to 400 mg of CHF 5074.

4. The pharmaceutical composition of claim 1, further comprising at least one additional neuroprotective agent.

5. The pharmaceutical composition of claim 4, wherein said neuroprotective agent is selected from the group consisting of (1) an Aβ peptides level reducer, (2) a pathogenic level tau reducer, (3) a microtubule stabilizer, (4) an agent capable of removing atherosclerotic plaques, (5) an agent that lower circulating level of β-amyloid and tau, (6) a modulator of autophagy, (7) a neurotransmitter level regulator, (8) a GABA(A) α5 receptor antagonist, (9) an additional agent that helps maintain and/or restores cognitive function and functional deficits of AD, and/or slows down decline in cognitive functions and functional deficits in AD, and (10) a mixture thereof.

6. The pharmaceutical composition of claim 5, wherein said Aβ peptides level reducer is selected from the group consisting of an agent inhibiting synthesis of APP, an agent that prevents formation of Aβ peptides, an inhibitor of mGlu2/3 auto-receptor, an alpha-secretase modulator, a beta-secretase inhibitor, a gamma-secretase inhibitor, a gamma-secretase modulator, a 5-HT4 agonist, an antibody to Aβ peptides and β-amyloid, an immunogenic peptide that results in the production of antibodies to β-amyloid, a blocker of oligomers' aggregation, a fibril formation inhibitor, a RAGE antagonist, and a mixture thereof.

7. The pharmaceutical composition of claim 4, wherein said neuroprotective agent is a metal protein interaction-attenuating compound, an activator of Sirtuin proteins, an HDAC inhibitor, or a combination of any two or more of the foregoing.

8. The pharmaceutical composition of claim 7, wherein the activator of Sirtuin proteins is resveratrol.

9. A method of treatment for the prevention or therapeutical treatment of proteinopathies and/or neurodegenerative diseases, including delaying the onset, slowing the progression or ameliorating symptoms of these diseases, comprising administering a 1-phenylalkanecarboxylic acid before, after, or concurrently with at least one additional neuroprotective agent to a mammal, in need of thereof.

10. The method of claim 9, wherein the 1-phenylalkanecarboxylic acid is CHF 5074.

11. The method of claim 10, wherein CHF 5074 is administered in a daily dosage amount from about 50 mg to about 550 mg of CHF 5074.

12. The method of claim 10, wherein CHF 5074 is administered in a daily dosage amount from 200 mg to 400 mg.

13. The method of claim 9, wherein said neuroprotective agent is selected from the group consisting of (1) an Aβ peptides level reducer, (2) a pathogenic level tau reducer, (3) a microtubule stabilizer, (4) an agent capable of removing atherosclerotic plaques, (5) an agent that lower circulating level of β-amyloid and tau, (6) a modulator of autophagy, (7) a neurotransmitter level regulator, (8) a GABA(A) α5 receptor antagonist, (9) an additional agent that helps maintain and/or restores cognitive function and functional deficits of AD, and/or slows down decline in cognitive functions and functional deficits in AD, and (10) a mixture thereof.

14. The method of claim of claim 13 wherein said Aβ peptides level reducer is selected from the group consisting of an agent inhibiting synthesis of APP, an agent that prevents formation of Aβ peptides, an inhibitor of mGlu2/3 auto-receptor, an alpha-secretase modulator, a beta-secretase inhibitor, a gamma-secretase inhibitor, a gamma-secretase modulator, a 5-HT4 agonist, an antibody to Aβ peptides and β-amyloid, an immunogenic peptide that results in the production of antibodies to β-amyloid, a blocker of oligomers' aggregation, a fibril formation inhibitor, a RAGE antagonist, and a mixture thereof.

15. A combination therapy for the treatment of one or more proteinopathies and/or neurodegenerative diseases, including delaying the onset, slowing the progression or ameliorating symptoms of these diseases, comprising administering to a mammal in need thereof a therapeutically effective dose of 1-phenylalkanecarboxylic acid, its pro-drug, or a bioisoster on the carboxylic moiety together with a therapeutically effective amount of one or more additional neuroprotective agents selected from the group consisting of: (1) an Aβ peptides level reducer, (2) a pathogenic level tau reducer, (3) a microtubule stabilizer, (4) an agent capable of removing atherosclerotic plaques, (5) an agent that lower circulating level of β-amyloid and tau, (6) a modulator of autophagy, (7) a neurotransmitter level regulator, (8) a GABA(A) α5 receptor antagonist, (9) an additional agent that helps maintain and/or restores cognitive function and functional deficits of AD, and/or slows down decline in cognitive functions and functional deficits in AD, and (10) a mixture thereof.

16. The combination therapy of claim 15, wherein said 1-phenylalkanecarboxylic acid is orally administered.

17. The combination therapy of claim 15, wherein said 1-phenylalkanecarboxylic acid is CHF 5074, and is administered in a daily dosage amount of from about 50 mg to about 550 mg.

18. The combination therapy of claim 15, wherein said 1-phenylalkanecarboxylic acid is CHF 5074, and is administered in a daily dosage amount of from about 200 mg to about 400 mg.

19. The combination therapy of claim 15, wherein said 1-phenylalkanecarboxylic acid and said additional neuroprotective agent are administered simultaneously.

20. The combination therapy of claim 15, wherein said 1-phenylalkanecarboxylic acid and said additional neuroprotective agent are administered sequentially.

21. The pharmaceutical composition of claim 5, wherein said 1-phenylalkanecarboxylic acid is conjugated to an antibody.

22. The pharmaceutical composition of claim 5, wherein said 1-phenylalkanecarboxylic acid is CHF 5074 which is chemically linked to an amyloid-clearing antibody.

23. A method of delaying the onset, slowing the progression or ameliorating symptoms of one or more proteinopathies and/or neurodegenerative diseases, comprising administering to a human patient in need thereof a therapeutically effective dose of a 1-phenylalkanecarboxylic acid before, after, or together with a therapeutically effective amount of a neuroprotective agent selected from the group consisting of (1) an Aβ peptides level reducer, (2) a pathogenic level tau reducer, (3) a microtubule stabilizer, (4) an agent capable of removing atherosclerotic plaques, (5) an agent that lower circulating level of β-amyloid and tau, (6) a modulator of autophagy, (7) a neurotransmitter level regulator, (8) a GABA(A) α5 receptor antagonist, (9) an additional agent that helps maintain and/or restores cognitive function and functional deficits of AD, and/or slows down decline in cognitive functions and functional deficits in AD, and (10) a mixture thereof, as part of a combined treatment regimen.

24. The method of claim 23, wherein said neuroprotective agent is an antibody that discriminates between an Aβ peptide and the β-amyloid protein precursor from which it is proteolytically derived.

25. The method of claim 24, wherein said antibody is end-specific and generated from an immunogenic peptide incorporating either a free N-terminus or a free C-terminus of an amyloid β-peptide involved in pathogenesis of Alzheimer's disease.

26. The method of claim 23, wherein said neuroprotective agent is an isolated antibody.

27. The method of claim 23, wherein said neuroprotective agent is an isolated antibody capable of selectively recognizing prefibrillar pathological or neurotoxic tau, including their pathogenic conformations.

28. The method of claim 27, wherein said antibody has an equilibrium constant KD with the antigen it is selective for of from 1×10⁻⁹ M to 1×10⁻¹¹ M in-vitro; and has an equilibrium constant KD with other peptides or proteins which is from 1×10⁻⁴ M to 1×10⁻⁶ M or shows no detectible binding or reactivity with these other peptides or proteins in-vitro, when tested at the saturating level of antibody-immunogen binding using 0.1 μg/nil of the antibody on a dot blot with 50 ng of the peptide or protein.

29. A method of increasing efficacy and decreasing side effects associated with a therapeutic agent used for the treatment of AD, said method comprising administering to a human patient in need thereof such treatment a therapeutically effective dose of a 1-phenylalkanecarboxylic acid before, after, or together with a therapeutically effective amount of a neuroprotective agent to augment the effect of said 1-phenylalkanecarboxylic acid, wherein said neuroprotective agent is selected from the group consisting of (1) an Aβ peptides level reducer, (2) a pathogenic level tau reducer, (3) a microtubule stabilizer, (4) an agent capable of removing atherosclerotic plaques, (5) an agent that lower circulating level of β-amyloid and tau, (6) a modulator of autophagy, (7) a neurotransmitter level regulator, (8) a GABA(A) α5 receptor antagonist, (9) an additional agent that helps maintain and/or restores cognitive function and functional deficits of AD, and/or slows down decline in cognitive functions and functional deficits in AD, and (10) a mixture thereof, as part of a combined treatment regimen.

30. A method of modulating microglial phagocytic activity, said method comprising administering to a human patient in need thereof a therapeutically effective amount of a 1-phenylalkanecarboxylic acid to prevent or slow down microglial inflammatory activity before, after, or together with an effective amount of one or more additional neuroprotective agents to modulate the microglial phagocytic activity, wherein said neuroprotective agent is selected from the group consisting of (1) an Aβ peptides level reducer, (2) a pathogenic level tau reducer, (3) a microtubule stabilizer, (4) an agent capable of removing atherosclerotic plaques, (5) an agent that lower circulating level of β-amyloid and tau, (6) a modulator of autophagy, (7) a neurotransmitter level regulator, (8) a GABA(A) α5 receptor antagonist, (9) an additional agent that helps maintain and/or restores cognitive function and functional deficits of AD, and/or slows down decline in cognitive functions and functional deficits in AD, and (10) a mixture thereof, as part of a combined treatment regimen.

31. A method of modulating microglial inflammatory activity, said method comprising administering to a human patient in need thereof a therapeutically effective amount of a 1-phenylalkanecarboxylic acid to prevent or slow down microglial inflammatory activity before, after, or together with an effective amount of one or more additional neuroprotective agents to modulate the microglial inflammatory effect, wherein the neuroprotective agent is selected from the group consisting of (1) an Aβ peptides level reducer, (2) a pathogenic level tau reducer, (3) a microtubule stabilizer, (4) an agent capable of removing atherosclerotic plaques, (5) an agent that lower circulating level of β-amyloid and tau, (6) a modulator of autophagy, (7) a neurotransmitter level regulator, (8) a GABA(A) α5 receptor antagonist, (9) an additional agent that helps maintain and/or restores cognitive function and functional deficits of AD, and/or slows down decline in cognitive functions and functional deficits in AD, and (10) a mixture thereof, as part of a combined treatment regimen.
